(19) United States
(12) Patent Application Publication
(10) Pub. No.: US 2004/0023296 A1
(43) Pub. Date: Feb. 5, 2004

(54) Use of quantitative evolutionary trace analysis to determine functional residues

(75) Inventor: Olivier Lichtarge, Bellaire, TX (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine

(21) Appl. No.: 10/306,495

(22) Filed: Nov. 27, 2002

Related U.S. Application Data
(60) Provisional application No. 60/333,796, filed on Nov. 28, 2001.

(51) Int. Cl. G01N 33/53; G06F 19/00; G01N 33/48; G01N 33/50

(52) U.S. Cl. 435/7.1; 702/19

(57) ABSTRACT

The present invention relates to methods to determine functional sites of a sequence using quantitative ET analysis. More particularly, the quantitative ET analysis utilizes gap tolerance and clustering statistics to determine the functional sites.

This application claims priority to U.S. Provisional Application serial No. 60/333,796 filed on Nov. 28, 2001.

This invention was made with government support under NSF Grant No. DBI-01 14796 awarded by the National Science Foundation. The United States Government may have certain rights in the invention.

### BACKGROUND OF THE INVENTION

I. Field of Invention

The present invention relates to structural biology and molecular engineering. More particularly, the present invention relates to determining functional residues using quantitative evolutionary trace analysis.

II. Related Art

As the production of putative genes from whole genome shotgun sequencing continues to grow exponentially, the Structural Genomics Initiative (SGI) aims to produce an equally daunting number of new protein structures, thus raising an important question as to how this mass of raw data is transformed into biologically meaningful information.

Determining functional sites on the surface of a protein is not trivial. The best approach to date is mutational analysis whereby individual residues are altered one at a time and the mutant protein's various functions are subsequently assayed. This is slow and costly and, critically, it also requires that accurate assays of each of the protein's multi-faceted functions be available. This requirement is problematic since the functions of many proteins are either not known or are only partially known. Nevertheless, elegant studies have shown that this strategy is highly successful at delineating functional hot spots, and reveal that these are often a smaller subset of the entire set of interfacial contact residues seen in crystal complexes (Pearce et al., 1996).

As an alternative to the protein-specific nature of mutational analysis, a number of laboratories have sought general computational approaches to characterize functional sites. Analyses that seek motifs, either based on complementarity of shape (Kuntz et al., 1982) or of charge (Honig et al., 1995), on empirical energy functions (Miranker et al., 1991), including the energetic links between proteins and ligands (Lamb et al., 1997) or the energetic effect of substitutions (Reyes et al., 2000), are all best done in the setting of an already identified binding partner. A second set of methods seeks to predict functional surfaces de novo. Casari et al. developed an algebraic method termed principal component analysis that treats proteins as vectors in a sequence space (Casari et al., 1995). Henikoff & Henikoff seek block-like motifs and then measure the chance distribution of their matches against the SWISS-PROT database (Henikoff et al., 1991). Jones and Thornton use physico-chemical descriptors of surface residues to score their probability of interacting in protein-protein interactions (Jones et al, 1997) based on a database of interfaces. Shatsky and colleagues align hinge regions and flexible regions of proteins by detecting maximal congruent rigid fragments so as to obtain their optimal arrangement and to identify functional interfaces (Shatsky et al., 2000). At their core, most of these methods focus on measuring global aspects of residue conservation as a marker of importance.

Another approach is Evolutionary Trace (Lichtarge et al., 1996a; Lichtarge et al., 1996), which combines both sequence and structure information to identify the location and specificity determinants of functional sites in homologous proteins. This is immediately useful because functional surfaces mediate all protein-ligand interactions. Knowledge of their location and specificity determinants helps target mutagenesis to the relevant residues of a protein in order to understand the molecular basis of function (Sowa et al., 2000; Sowa et al., 2001) and it is also useful for drug design and for engineering new, desirable properties into proteins (Ma et al., 2001).

Evolutionary Trace (ET) has been useful in identifying some functional surfaces, however, gaps in sequences cause problems for the method. Insertions and deletions in sequences creates a dilemma, which results in a compromise to selectively remove sequences that introduce the gaps in the alignment. Thus, ET is merely a qualitative analysis of protein structure.

Another disadvantage of ET is that ET requires visual interpretation of its results. The user must recognize, by eye, clusters of top-ranked residues in 3D space and visually estimate their significance based on the level of scattered signal throughout the protein. A few large clusters are interpreted as true signal, while many small clusters scattered homogeneously about the protein indicate noise. Although this evaluation is fairly straightforward, it is also subjective, especially near the signal-to-noise threshold, which is the region of ranks were the 3-dimensional clustering behavior of trace residues in the structure becomes indistinguishable from that of randomly picked residues.

Thus, there is a need to develop a method that is able to handle gaps and one that is also objective in determining clusters. Yet further, a need also exists for a method that aligns sequences with remote homologies and determines global functional sites in families of structures. Still further, there is a need to develop a quantitative method of determining functional sites accurately in a diverse set of proteins.

### BRIEF SUMMARY OF THE INVENTION

The present invention is directed to a method which quantitatively determines a functional site in a protein. The present invention introduces a novel gap tolerant trace, by adopting the convention that gaps are a virtual twenty-first amino acid type. Yet further, to replace human assessment, the present invention uses quantification methods to provide an objective measure of clusters. One example of a quantification method is statistics. The statistics includes the overall number of clusters and the size of the largest cluster. Random sampling of residues in several structures allows the distributions of these statistics to be used to provide an estimate and to measure the significance of the actual quantitative ET generated values. Once the functional site of the structure is determined, then the site is used for rational drug design and/or protein engineering. Yet further, the quantitative ET generated values are used to model the quaternary structure or to integrate sequence and structure databases to extract information on the molecular basis of the function. The present invention can also be used to assess whether the 3-dimensional clusters of trace residues are consistent with an evolutionary bias that indicates a functional site rather than a random process.

A specific embodiment of the present invention is a method of determining a functional site in a protein comprising the steps of: obtaining a protein sequence; aligning the protein sequence to homologous protein sequences to generate a multiple sequence alignment; adding gap tolerance, wherein a gap in the protein sequence alignment is considered as an artificial amino acid; producing an evolutionary trace, wherein the evolutionary trace identifies residues that are trace residues; and determining cluster formation of the trace residues, wherein a cluster indicates the functional site of the protein. In further embodiments of the present invention, the method also comprises determining clustering statistics. It is envisioned that the method of the present invention is further used to assess the significance of a single nucleotide polymorphism (SNP) providing that the SNP is located within or near a functional site. Yet further, it is envisioned that the method of the present invention is used for any sequence, for example, nucleic acid sequences or amino acid sequences.

Another embodiment of the present invention is a protein database. The protein database comprises proteins having predicted functional sites. It is envisioned that the database is produced using quantitative evolutionary trace analysis having gap tolerance and/or clustering statistics. The method of producing a protein database having predicted functional sites comprises the steps of obtaining a protein sequence; aligning the protein sequence to homologous protein sequences to generate a multiple sequence alignment; adding gap tolerance, wherein a gap in a protein sequence alignment is considered as an artificial amino acid; producing an evolutionary trace, wherein the trace identifies residues that are trace residues; and determining cluster formation of the residues, wherein a cluster indicates the functional site of the protein.

Yet further, another embodiment of the present invention is a peptide database comprising peptides, which are the binding sites of proteins. It is envisioned that the peptide database is produced using quantitative evolutionary trace analysis having gap tolerance and/or clustering statistics. The method of producing a peptide database having peptides that are binding sites of proteins comprises the steps of obtaining a peptide sequence; aligning the peptide sequence to homologous peptide sequences to generate a multiple sequence alignment; adding gap tolerance, wherein a gap in a peptide sequence alignment is considered as an artificial amino acid; producing an evolutionary trace, wherein the trace identifies residues that are trace residues; and determining cluster formation of the residues, wherein a cluster indicates the binding site.

Another embodiment of the present invention is a method of aligning remote homologs. The method comprises the steps of obtaining protein sequences of at least two proteins with no sequence homology; producing a separate evolutionary trace sequence of each protein, wherein the evolutionary trace sequence identifies residues that are trace residues; assigning evolutionary rank to trace residues from each trace; assigning an order based on the evolutionary rank; determining a correlation between any two trace residues, wherein a correlation of greater than zero indicates that the trace residues have evolutionary ranks that are dependent on each other and a correlation of zero indicates that the trace residues have evolutionary ranks that are independent of each other; aligning the traces from the protein sequence, wherein aligning is performed to maximize the evolutionary rank order correlation from each trace; and determining a correlation between the two proteins with no sequence homology.

A specific embodiment is a method of determining a ligand binding pocket in a protein comprising the steps of: determining global functional determinates of a family of proteins using quantitative evolutionary trace analysis, wherein determinates are residues that are involved in the global function of the protein; obtaining protein sequences of a subfamily of proteins within the family having a common function; aligning the protein sequences of the subfamily of proteins to generate a multiple sequence alignment; producing an evolutionary trace, wherein the evolutionary trace identifies residue that are trace residues; and comparing the evolutionary trace of the family to the evolutionary trace of the subfamily, wherein a difference in the comparison yields the ligand binding pockets of the protein.

Another embodiment of the present invention is a method of designing pharmaceuticals that target a protein comprising the steps of: obtaining a protein sequence; aligning the protein sequence to homologous protein sequences to generate a multiple sequence alignment; predicting at least one residue in the protein sequence which is involved in the protein's function, wherein predicting the residues involves using quantitative evolutionary trace analysis; and synthesizing the pharmaceutical to interact with the predicted residue in the protein. It is envisioned that the pharmaceutical is a protein, a peptide or small molecule. Yet further, the method can also comprise mutating at least one predicted residue prior to synthesizing the pharmaceutical. It is contemplated that the mutation of at least one predicted residue results in modulation of the protein's function, wherein modulation is an enhancement or an interference with the protein's function, for example, binding of the protein to it's target or receptor. Thus, the mutation of at least one residue can result in an antagonist or an agonist pharmaceutical.

Yet further, another embodiment of the present invention is a method to design proteins that have desired and/or altered protein properties. The method may comprise the steps of: obtaining a protein sequence; aligning the protein sequence to homologous protein sequences to generate a multiple sequence alignment; predicting at least one residue in the protein sequence which is involved in the protein's functions, wherein predicting the residue involves using quantitative evolutionary trace analysis; synthesizing libraries of protein variants wherein residues at and/or around the predicted functional site are substituted with alternative amino acids; and screening the resulting libraries for mutant proteins with the desired protein properties.

Yet further, another embodiment is a method to identify residues on a protein structure that are least likely to be part of a functional site that is critical to biochemical activity, binding, or structure folding and stability. The residues are identified using quantitative evolutionary trace analysis. Such residues can be targeted for mutation to impart altered protein properties to the protein without destroying the protein's fold, structure, and normal function. Thus, it is envisioned that the mutations can result in a variety of altered protein properties (e.g., increase and/or decrease), for example, but not limited to binding affinity, aggregation, crystallization, solubility, stability (e.g., degradation, post-translational modification), immunogenicity, and other properties that are part of the protein's normal biological, cellular, and physico-chemical behavior and effects. Yet further, depending upon the desired protein property, the residues can be mutated to enhance and/or decrease any of the desired protein properties.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the Evolutionary Trace method.

FIG. 2 shows the sequence identity tree of Gα submits. The sequences of 88 G-protein α-subunits were retrieved from SwissProt and aligned using the GCG program PILEUP and the resulting dendrogram is shown as the dashed line, which indicates the tree partition where the five principal G α subgroups are separated.

FIG. 3 shows the ET prediction and mutational analysis agree on GPRC binding sites on Gα. There is agreement at 74 residues, shown in black. 17 residues were false negatives, at this functional resolution (in medium gray), and 17 were false positives (in light gray). Nt. Amino-terminus of Gα.

FIG. 4 shows a model of rhodopsin/G-protein binding. The receptor is shown in gray and is oriented in a cartoon of the lipid bilayer with the intracellular space oriented at the top of the figure. The G protein is colored as follows: Gα, white; Gβ, gray; Gγ white. Black denotes trace residues on all four proteins.

FIG. 5A, FIG. 5B and FIG. 5C show an ET analysis of the RGS family and reveals two distinct active sites. FIG. 5A shows the Gα interaction surface on the RGS domain is correctly identified by ET (region on Gα that principally contacts the RGS domain is shown as secondary structure) and is composed of 4 invariant and 6 class-specific residues (only 1 contact residue was not identified by ET at this resolution, rank=20). A second evolutionarily privileged site, term R2, is located in close proximity to the RGS/Gα catalytic interface but does not directly contact Gα. FIG. 5B shows the complex of RGS4-G₁ₗα GDP A1F₄, which shows R2 is exposed above the RGS/Gα interface and could function as a binding site for other factors to bind and modulate RGS activity. FIG. 5C shows the ternary complex of the RGS9-1 core domain, G_{t/il}α GDP A1F₄, and the C-terminal 38 amino acids from the effector subunit PDEγ, reveals that the effector binds Gα along site R2 with which it contacts at residues 360 and 362.

FIG. 6 shows the mutational analysis of the RGS domain. The proteins were then assayed for their ability to increase the rate of GTP hydrolysis by Gₜαin either the absence (black bars) or presence (hashed bars) of PDE.

FIG. 7A and FIG. 7B show the GPCR correlation. Spearman rank-order correlation coefficients are shown for the comparison between the opsin and adrenergic receptors as alignments are shifted by n residues (FIG. 7A). Correlation coefficients are shown for Class A versus Class B comparisons (FIG. 7B).

FIG. 8 shows global and ligand specificities in GPCRs. Comparison between residues in the bottom 15^{th} rank-order percentile from the visual opsin family and from selected receptors from [Class A+Class B], shown in the rhodopsin structure. Residues that are unique to opsins, in white, form a cluster around the retinal moiety with a narrow extension toward the G protein. This extension then mushrooms into a network of interaction that involves residues from all TMs and that extends to the intracellular loops. These residues, in gray, are important to both the opsins and the other members of Class and B included in this analysis. A few residues, in black, are in the bottom 15^{th} rank percentile for [Class A+Class B] receptors, but not for opsins.

FIG. 9A, FIG. 9B, FIG. 9C, FIG. 9D, FIG. 9E, FIG. 9F, FIG. 9G, and FIG. 9H show residues identified by ET cluster non randomly in pyruvate decarboxylase. Trace residues tend to form a small number of large clusters (FIG. 9A-FIG. 9D are rotated with respect to each other by 90° about the y-axis), while an equivalent number of randomly selected residues form many small clusters scattered homogeneously throughout the protein (FIG. 9E-FIG. 9H are rotated in the same manner as FIG. 9A-FIG. 9D). The trace residues shown correspond to those identified at rank 10, or 20% coverage of the protein (PDB identifier: 1pvd), where 90 residues are predicted to be important by ET.

FIG. 10A and FIG. 10B show the random distribution of the expected number of clusters are used to establish significance thresholds. FIG. 10B shows the linear relationship between protein size and the number of clusters predicted by random simulations. Each point represents 5000 random simulations performed on a different protein (12 proteins in all) at 15% coverage with a significance threshold of 1%.

FIG. 11A, FIG. 11B, FIG. 11C, FIG. 11D, FIG. 11E and FIG. 11F show a significance of ET predictions using the ‘Number of Clusters’ statistics. For 10%, 20%, and 30% coverages, the number of clusters identified by ET was plotted against protein size for each of the 46 proteins with a rank directly convertible to a coverage level. “Trace With Gaps” (FIG. 11A-FIG. 11C) refers to ET data generated when considering gaps in the alignment and “Trace Without Gaps” (FIG. 11D-FIG. 11F) refers to the ET data generated without this information. The significance thresholds are shown as: 0.3%=dark gray line; 5%=black line; 30%=light gray line and were generated using the linear fits from the number of clusters simulations.

FIG. 12A and FIG. 12B show the size of the largest cluster. Similar to the number of clusters study, the linear relationship between protein size and the size of the largest cluster predicted by random simulations is shown in the FIG. 12B. Each point represents 5000 random simulations performed on a different protein (12 proteins in all) at 20% coverage with a significance threshold of 0.3%.

FIG. 13A, FIG. 13B, FIG. 13C, FIG. 13D, FIG. 13E and FIG. 13F show the significance of ET predictions using the ‘Size of Largest Cluster’ statistics. For 10%, 20%, and 30% coverages, the size of the largest cluster predicted by ET is plotted against protein size for each of the 46 proteins with a rank directly convertible to a coverage level was. “Trace With Gaps” (FIG. 13A-FIG. 13C) refers to ET data generated when considering gaps in the alignment and “Trace Without Gaps” (FIG. 13D-FIG. 13F) refers to the ET data generated without this information. The significance thresholds are shown as: 0.3%=dark gray line; 5%=black line; 30%=light gray line and were generated using the linear fits from the size of the largest cluster simulations.

FIG. 14A, FIG. 14B, FIG. 14C, FIG. 14D, FIG. 14E, FIG. 14F, FIG. 14G and FIG. 14H show ET clusters overlap with known ligand binding domains. In the representative cases of 2,5-diketo-d-gluconic acid reductase A (1a80, FIG. 14A-FIG. 14C) and dihydropteroate synthase (1aj2, FIG. 14D-FIG. 14F), the structural epitopes, defined as all the residues within 5 Å of the ligand (shown in white), are shown in gray (FIG. 14A, FIG. 14D). ET-identified residues surround and include residues from the structural epitopes for both proteins when gaps are excluded (FIG. 14B at rank 66, FIG. 14E at rank 13) and included (FIG. 14C at rank 55, FIG. 14F at rank 23) from the ET analysis. Individual ET-identified residue clusters are shown in gray, medium gray, and black (FIG. 14A-FIG. 14C) and in gray and light gray (FIG. 14E-FIG. 14F). In the case of 1a80, the clustering pattern is noticeably different when gaps are included or excluded from the analysis. This is due to the fact that when gaps are excluded, the rank at which the structural epitope is identified is greater than when gaps are included (compare FIG. 14B to FIG. 14C). As rank increases, separate small clusters tend to coalesce into larger clusters (compare FIG. 14C to FIG. 14B). These ET identified residues do not overlap completely with the structural epitope consistent with the fact that not all of the residues in the binding site contribute to ligand interaction.

FIG. 15A, FIG. 15B, FIG. 15C and FIG. 15D show overlap statistics. The protein is gray and its functional site is delineated by the stripped white region. Trace residues are the small circles and they form trace clusters, outlined in black lines. Trace residues that meet the criteria set by the illustrated statistic used to measure the overlap between trace clusters and the functional site are filled in black, or left white otherwise. (FIG. 15A) “Total Connected Residues” counts as positive all residues connected to the functional site (in this case, 19) and as negative the rest. (FIG. 15B) “Largest Cluster Overlap” only counts as positive the residues shared by the largest cluster and by the functional site. (FIG. 15C) “Average Overlap” averages all overlapping trace residues by the number of overlapping trace clusters (8/2). (FIG. 15D) “Hypergeometric Distribution” counts as positive any trace residue in the functional site, regardless of their clustering properties.

FIG. 16A and FIG. 16B show trace clusters overlap significantly with functional sites. FIG. 16A shows the fraction of manually optimized traces (black) or automated traces (white) that significantly overlap with functional sites for at least one rank, is shown for each statistic: Total Connected Residues (TCR), Largest Cluster Overlap (LCO), Average Overlap (AO), and Hypergeometric Distribution (HD). FIG. 16B shows the fraction of trace ranks with significant clusters that also significantly overlap the functional site. This is averaged for each dataset.

FIG. 17A, FIG. 17B, FIG. 17C, FIG. 17D, FIG. 17E and FIG. 17F show the largest significant trace cluster overlaps most of the functional site. This is shown for both manually refined traces in (FIGS. 17A-17C) and automated traces in panels (FIGS. 17D-17F). The overlap is especially extensive in the enzyme set where the sites are small, but it is also extensive in the sites defined by the approximate criterion of ligand proximity, often covering more than 50% of the site, even with the automated traces.

FIG. 18 shows the Quantitative Evolutionary Trace method.

### DETAILED DESCRIPTION OF THE INVENTION

It is readily apparent to one skilled in the art that various embodiments and modifications may be made to the invention disclosed in this application without departing from the scope and spirit of the invention.

As used herein the specification, “a” or “an” may mean one or more. As used herein in the claim(s), when used in conjunction with the word “comprising”, the words “a” or “an” may mean one or more than one. As used herein “another” may mean at least a second or more.

As used herein, the term “aggregation” refers to the interaction of proteins, usually non-specific, to form a complex that may or may not be covalently linked.

As used herein, the term “agonist” is defined as a substance that has an affinity for the active state of a receptor and thereby preferentially stabilizes the active state of the receptor or a compound, including, but not limited to, proteins, peptides, nucleic acids, pharmaceuticals, hormones and neurotransmitters, which produces activation of receptors. Irrespective of the mechanism(s) of action, an agonist produces activation of receptors.

As used herein, the term “antagonist” is defined as a substance that does not preferentially stabilized either form of the receptor, active or inactive, or a compound, including, but not limited to, proteins, peptides, nucleic acids, pharmaceuticals, hormones and neurotransmitters, which prevents or hinders the effects of agonists and/or inverse agonists. Irrespective of the mechanism(s) of action, an antagonist prevents or hinders the effects of agonists and/or agonists.

As used herein, the term “inverse agonist” is defined as a substance that has an affinity for the inactive state of a receptor and thereby preferentially stabilizes the inactive state of the receptor, or a compound, including, but not limited to, proteins, peptides, nucleic acids, pharmaceuticals, hormones and neurotransmitters, which produces inactivation of receptors and/or prevents or hinders activation by agonists. Irrespective of the mechanism(s) of action, an inverse agonist produces inactivation of receptors and/or prevents or hinders activation by agonists.

As used herein, the term “alternative hypothesis” or “H₁” refers to the hypothesis that in some sense contradicts the null hypothesis.

As used herein, the term “class specific residue” refers to a residue that has a position that is conserved within each group or class, but among the group of residues has a different identity. Thus, one skilled in the art realizes that class specific residues are invariant within functional classes but variable among them. Yet further, these class specific residues impart unique functions to the proteins and/or DNA or RNA molecules in the family.

As used herein, the term “cluster” refers to a 3-dimensional geometric property whereby all the residues of a common cluster are within a distance of 4 Å of at least one other member of the cluster. This distance, 4 Å, is a parameter of the method that may be adjusted by the user, and it is measured from any non-hydrogen atom in one residue to any non-hydrogen atom in the other residue that is in the same cluster. Such clusters can be calculated at each rank, k, by mapping onto the structure of all trace residues of rank k, k−1, k−2, . . . , 3, 2, 1.

As used herein, the term “coverage” is defined as a fraction which is the number of trace residues at and above a given rank k (k, k−1, k−2, . . . , 3, 2, 1), divided by the total number of trace residues at the maximum possible rank.

As used herein, the term “database” is defined as a collection of sequences having predicted functional sites. Yet further, a database can comprise peptides that are known binding and/or functional sites.

As used herein, the term “dendrogram” is defined as a tree or binary branching diagram representing a hierarchy of categories defined by the branches based on degree of similarity or number of shared characteristics. Thus, the dendrogram of the present invention is a sequence identity tree that is closely related but not necessarily exactly the same as an evolutionary tree that details the ancestral relationships between the various sequences. A basic methodological assumption is that at each node in the tree, the sequences in either one of the daughter branches are more functionally similar to each other than they are to any sequence in the other daughter branch. The tree thereby provides from tree root to tree leaf an increasingly fine functional classification of the sequences.

As used herein, the term “rank” is defined as the number of separate groups considered for ET analysis. At rank k, there are k groups, each containing the sequence that are respectively contained in the first k branches of the tree, where counting starts from the root of the tree. Thus at rank 1, there is only one group containing all the sequences. At rank 2, the tree was used to separate these sequences into those from the first two branches, and so forth.

As used herein, the term “Evolutionary Trace” or “ET” refers to a method that identifies local patterns of conservation and global patterns of variation that intrinsically indicate functional or structural importance. This method uses a phylogenetic, or sequence identity, or any other reasonable tree either derived from a multiple sequence alignment, or constructed by the user as part of an experimental hypothesis, to approximate the functional clustering of family members. By partitioning the tree into distinct branches (deemed equivalent to functional classes), consensus sequences can be generated for each one and then compared. Residue positions that are invariant within each branch but variable among them are termed trace or class specific residues. By construction, these class specific residues are closely coupled to evolutionary divergence and hence, presumably, to functional importance. The minimum number of branches into which the tree has to be divided in order for a residue to become class specific is termed the “rank” of that residue. Top ranks (1, 2, 3, . . . ) indicate residues that have become fixed within each of the most ancient evolutionary clades of a family suggesting a fundamental link to function, whereas low ranks ( . . . , n−2, n−1, n; where n is the maximum number of sequences in the family) indicate residues that vary even among the most closely related of proteins suggesting they have little impact on function. Yet further, a residue of rank i is invariant within each of the first i branches of the tree (starting from the root), but variable within one of the first (i−1) branches. In certain embodiments of the method, there is strict invariance within the first i branches whenever a residues has rank i. In further embodiments of the method, user defined pre-determined substitutions can also be tolerated even within a branch. Such substitutions are determined by the user, for example, conservative substitutions can be defined and pre-determined by the user. Conservative substitutions are those that maintain a key distinguishing feature of an amino acid for example, a lysine can be substituted for arginine. Yet further, other substitutions by be defined by the user. It is contemplated that the user being skilled in the art is aware of substitutions that can be used and tolerated by the system, e.g., the user is aware of substations that will not interfere with the results of the method.

As used herein, the term “globular protein” refers to proteins in which their polypeptide chains are folded into compact structures. The compact structures are unlike the extended filamentous forms of fibrous proteins. A skilled artisan realizes that globular proteins have tertiary structures which comprise the secondary structure elements, e.g., helices, β sheets, or nonregular regions folded in specific arrangements. An example of a globular protein includes, but is not limited to myoglobin.

As used herein, the term “homolog” or “homologue” or “homologous” refers to a compound that has a similar likeness in structure. One skilled in the art realizes that the similarity often is attributable to the compounds having a common origin.

As used herein, the term “invariant residue” refers to a residue that has a position that is completely conserved across all family members. One skilled in the art realizes that invariant residues define the fundamental stereochemical architecture underlying activity of the molecule.

As used herein, the term “ligand” refers to a group, ion, or molecule coordinated to a central atom in a complex.

As used herein, the term “ligand binding pocket” refers to the structural location in a complex in which a group, ion or molecule binds.

As used herein, the term “library” or “combinatorial library” or “peptoids-derived library” and the like are used interchangeably herein to mean a mixture of organic compounds synthesized on a solid support from submonomer starting materials. Where the compounds of the library are peptoids, the peptoids can be cyclic or acyclic. The library will contain 10 or more, preferably 100 or more, more preferably 1,000 or more, and even more preferably 10,000 or more organic molecules which are different from each other (i.e., 10 different molecules and not 10 copies of the same molecule). Each of the different molecules will be present in an amount such that its presence can be determined by some means, e.g. can be isolated, analyzed, or detected with a receptor or suitable probe. The actual amount of each different molecule needed so that its presence can be determined will vary due to the actual procedure used and may change as the technologies for isolation, detection and analysis advance. When the molecules are present in substantially equal molar amounts an amount of 100 picomoles (pmol) or more can be detected.

As used herein, the term “mutation(s)” refers to a change of one or more amino acids in a protein. Mutations can include insertions, deletions or substitutions of amino acids. Yet further, one of skill in the art realizes that mutations can be produced by various known methodologies, for example, but not limited to chemical mutagenesis and/or molecular mutagenesis as described elsewhere in the present application. Thus, mutations of at least one amino acid residue results in a mutated protein as defined herein.

As used herein, the term “null hypothesis” or “H₀” refers to the hypothesis that is to be tested.

As used herein, the term “peptide” refers to a chain of amino acids with a defined sequence whose physical properties are those expected from the sum of its amino acid residues and there is no fixed three-dimensional structure.

As used herein, the term “protein” refers to a chain of amino acids usually of defined sequence and length and three dimensional structure. The polymerization reaction, which produces a protein, results in the loss of one molecule of water from each amino acid, proteins are often said to be composed of amino acid residues. Natural protein molecules may contain as many as 20 different types of amino acid residues, each of which contains a distinctive side chain.

As used herein, the term “protein function” refers to anyone of the many activities that allow a protein to perform its usual biochemical, cellular, physiological activity in its normal context. Such activities include, but are not limited to folding, cellular targeting, structural dynamics and stability, degradation kinetics as well as other interactions between the protein and the many molecules in its environment, and the transformations that it undergoes or effect as a result of these interactions.

As used herein, the term “protein properties” refers to a protein's normal biological, cellular, and physico-chemical behavior and effects. Exemplary protein properties include, but not limited to binding affinity, aggregation, crystallization, solubility, stability (e.g., degradation, post-translational modification), immunogenicity, etc. It is contemplated that protein properties can be either enhanced and/or decreased by the present invention. Thus, the enhanced and/or decreased property is a modulation of the protein property or alteration of the protein property.

As used herein, the term “residue” refers to a constituent structural unit of a complex molecule. For example, a residue refers to an amino acid of a protein. One skilled in the art is cognizant that a residue can also refer to a nucleic acid of a DNA or RNA molecule. Thus, a residue as used in the present invention refers to a structural unit, such as, an amino acid or a nucleic acid.

As used herein, the term “Quantitative Evolutionary Trace” or “QET” refers to an improved, quantitative method of ET which involves the quantification of cluster formation by any quantification method known and used by those of skill in the art.

As used herein, the term “solubility” refers to the amount of the protein that can be dissolved in a given volume of a solvent.

As used herein, the term “trace residue” refers to a residue that is class specific residue and/or invariant residue.

The characterization of active sites in proteins is an important problem in biology. Proteins carry out nearly all their functions through these specialized surfaces where the conformational, dynamic, and electrochemical properties of specific amino acids define which ligands they bind and which transformations are exerted on them. This preeminent role translates into widespread interest in active site identification and in elucidating how their constituent residues contribute to function. This is useful, for example, to design drugs or functional mimics to impart novel properties to engineered molecular scaffolds by transplanting these sites, and to develop generic biosensors. Moreover, a better understanding of active sites can help manipulate protein interactions and dissect cellular pathways.

The characterization of active sites or functional sites lags far behind the exponential growth of sequence and structure databases. One reason is that active sites can remain cryptic in protein structures that are determined without all their ligands. A deeper difficulty is that not all the interfacial residues contribute to an interaction. Thus, in order to determine the origin of affinity and specificity in molecular detail, it is necessary to perform mutational analysis. Unfortunately, constructing mutants and assaying activity are laborious tasks that are also protein specific and resource intensive, restricting their use to a fraction of all available protein structures.

By contrast, computational analysis is more amenable to large-scale application. Some algorithms, already usefully identify pockets or rough patches on the protein surface where small ligands preferentially bind, and electrostatic charge computations can yield clues to RNA or DNA binding sites. Predictions remain difficult, however, in the absence of recognizable geometric or chemical motifs. This is the case for the large interfaces typical in proteins from macromolecular cellular networks, which are indistinguishable from their surroundings based on hydrophobic character, solvation potential, planarity, protrusion, or accessible surface area.

The evolutionary trace method aims to facilitate active site characterization by combining an algorithmic approach with the experimental strategy of mutational analysis. It does so by categorizing natural sequence variations in terms of the evolutionary divergences of related proteins, thereby establishing an association between residue variation and functional changes.

I. Quantitative Evolutionary Trace Method

A specific embodiment of the present invention is a method of determining a functional site in a protein comprising the steps of: obtaining a protein sequence; aligning the protein sequence to homologous protein sequences to generate a multiple sequence alignment; adding gap tolerance, wherein a gap in a protein sequence alignment is considered as an artificial amino acid; producing an evolutionary trace, wherein the evolutionary trace identifies residues that are trace residues; and determining cluster formation of the trace residues, wherein a cluster indicates the functional site of the protein.

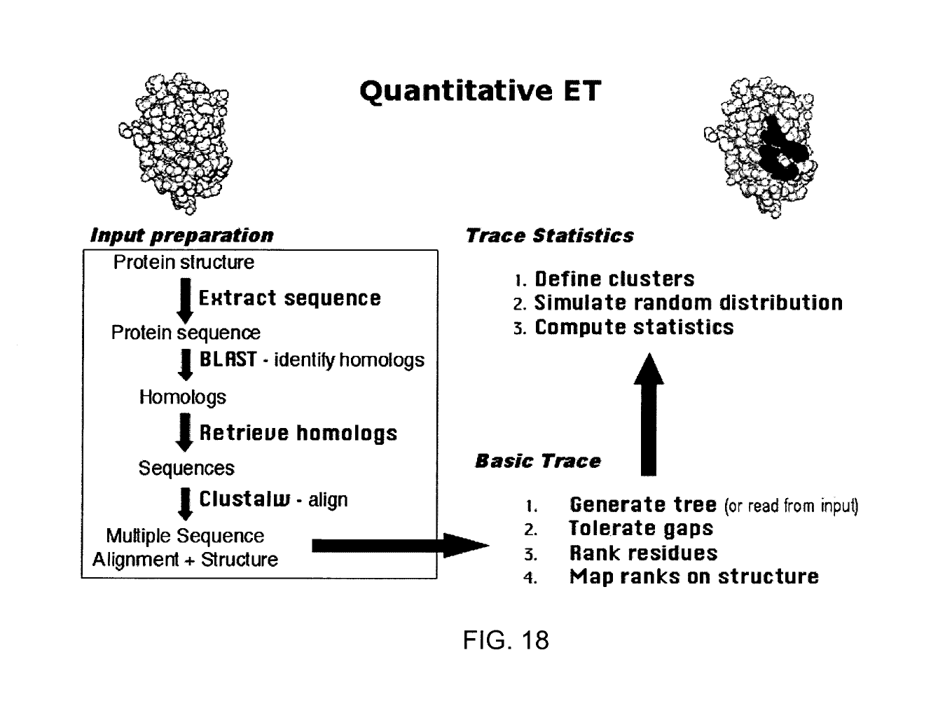

As shown in FIG. 18, the first step of QET is picking a target protein, e.g., protein of interest. Next, the sequence of the target protein is used to search (e.g., blast) databases and identify homologs of the protein. From the blast search, for example, a set of homologs is selected to define a hypothesis whereby it is assumed that all sequences in the homolog set perform a common function at a common structural site. Next, the sequences are aligned by any available alignment program, for example, e.g., CLUSTALW or PILEUP. Some of the sequences may have additional domains that are unrelated to the target sequence, thus these sequence portions that have no homology and no relation to the target sequence are normally removed, unless the user feels that they are important in their own right to further alter the nature of the multiple sequence alignment. QET automatically computes the sequence identity between the sequences of the multiple sequence alignment and builds a sequence identity dendrogram using a UPGMA method. Alternately, QET can automatically accept trees generated by the user from PILEUP, or PHYLIP. Once the residues are ranked by QET, the ranked residues are mapped onto any structure determined by NMR, x-ray, crystallography or modeling of a sequence in the set of homologs (since the sequences are homologs, their structures are closely related) or any other method of determining structures.

It is envisioned that the QET method of the present invention is used with any linear sequence, including, amino acid sequences or nucleic acid sequences. Nucleic acid sequences include, but are not limited to DNA and RNA.

Thus, specific embodiments of the present invention include a method of determining a functional site in a nucleic acid sequence comprising the steps of: obtaining a nucleic acid sequence; aligning the sequence to homologous sequences to generate a multiple sequence alignment; adding gap tolerance, wherein a gap in a sequence alignment is considered as an artificial nucleic acid; producing an evolutionary trace, wherein the evolutionary trace identifies nucleic acids that are trace nucleic acids; and determining cluster formation of the trace nucleic acids, wherein a cluster indicates the functional site of the nucleic acid sequence.

In specific embodiments, the sequence is RNA or ribonucleic acids. The use of QET to determine a functional site is beneficial to target pharmaceuticals at the ribosome, which is a combination of protein and RNA components and/or to target pharmaceuticals to RNA enzymes.

The sequences are obtained from databases, such as PDB, GenBank, or any other database that contains sequences. These databases are well known and used by those of skill in the art.

For the purposes of explaining the alignment of sequences, the sequences in which a functional site is to be determined are referred to as the target sequence. Thus, aligning of sequences in the present invention can involve aligning the target sequence to homologous sequences to generate a multiple sequence alignment, which is then used to construct a dendrogram. Preferably, the sequence alignment and dendrogram construction is performed with the GCG multiple sequence alignment tool PILEUP (Feng et al., 1987; Higgens et al., 1989). Yet further, it is envisioned that any well-known sequence alignment procedure can be used to perform the multiple sequence alignment and any well-known dendrogram construction procedure can be used to construct the dendrogram. For example, other sequence alignment procedures include, but are not limited to CLUSTALW (Thompson et al., 1994) and PHYLIP (Felsenstein, 1993), which provide sequence alignments and identity trees or dendrograms.

Another embodiment of the present invention is directed to a database of proteins and/or peptides having predicted functional sites and a method of developing such database. It is envisioned that the database is produced using quantitative evolutionary trace analysis having gap tolerance and/or clustering statistics. The method of producing a protein and/or peptide database having predicted functional sites comprises the steps of obtaining a protein and/or peptide sequence; aligning the protein and/or peptide sequence to homologous protein and/or peptide sequences to generate a multiple sequence alignment; adding gap tolerance, wherein a gap in a protein and/or peptide sequence alignment is considered as an artificial amino acid; producing an evolutionary trace, wherein the trace identifies residues that are trace residues; and determining cluster formation of the residues, wherein a cluster indicates the functional site of the protein and/or peptide.

A. Principles of Evolutionary Trace Method

The basic hypothesis of the ET method is that protein active sites evolve through variations on a conserved architecture. Active sites from divergent proteins are expected to have two evolutionary components: one that is invariant, and one that is specific to each functional class. Correspondingly, there are two types of functionally important residues. The first type is mostly invariant and thereby defines the fundamental stereochemical architecture underlying activity. The second type is invariant within functional classes but variable among them. These so-called class-specific residues impart unique functions to the proteins in the family.

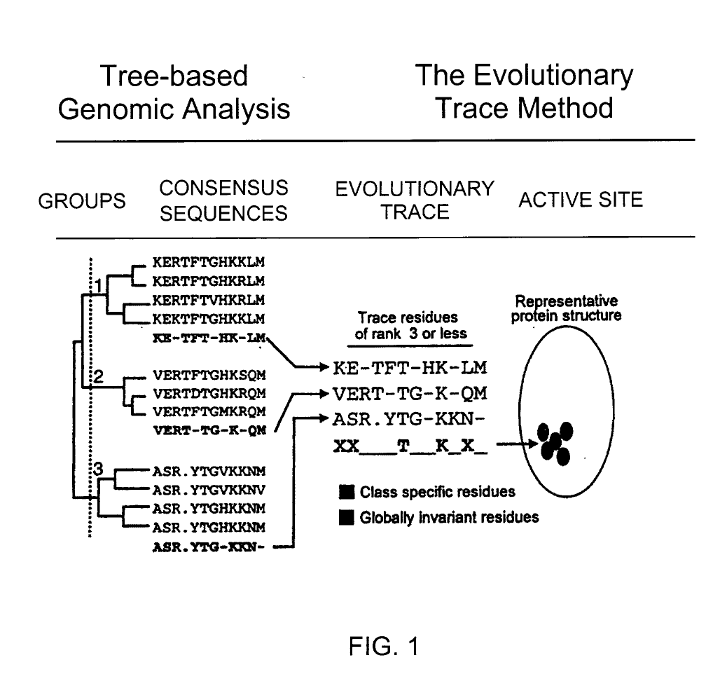

The model, as shown in FIG. 1, leads to a procedure to identify class specific residues. First, homologs of a protein of interest are gathered, aligned, and separated into functional subgroups so that the invariant residues of each group are identified in a consensus sequence. Second, these consensus sequences are compared to reveal positions that are invariant within each class but variable among them. These are the class-specific positions, or residues. By construction, their variations are always associated with a change in function, which is the sine qua non of functionally important residues. In the last step class-specific residues are mapped onto a representative structure of the protein family. If they cluster, this indicates an evolutionary privileged site where variations are strictly linked to functional differentiation, as would be expected from an active site.

Since database searches retrieve tens or even hundreds of related proteins or compounds whose functions have never been tested in the laboratory, a sequence identity tree is used as a good approximation of a functional classification. This is plausible because proteins with very similar sequences will have diverged relatively recently and should therefore have more closely related functions than proteins with weaker sequence similarity. In practice, sequence identity relationships provide a sensible estimate of functional relationships as is seen from the sequence identity tree (dendrogram) of Gα subclasses Gᵢα, Gₒα, Gₜα, Gₛα and G_{q}α into different branches.

B. Consequences of Tree Usage

In specific embodiments of the present invention, sequence alignment and dendrogram construction is performed. The present invention is not limited to a dendrogram representation. Other phylogenetic, evolutionary trees, or other data structures that detail the nature of the ancestral relationships between multiple sequences is used.

The use of a sequence identity tree has several advantages. First, it completes the remaining step in FIG. 1, so that the evolutionary trace is a fully defined algorithmic procedure. Examples of sequence alignment procedures, include, but are not limited to standard software such as PILEUP (distributed through GCG), or CLUSTALW (Thompson et al., 1994) and PHYLIP (Felsenstein, 1993), which provide sequence alignments and identity trees. Although these programs may not generate perfectly identical trees, most differences are confined to nodes that are near the leaves rather than the root of the tree. Since these terminal nodes contribute little to a trace, these variations have little impact. If sequence identity drops below 25-30%, however, nodes that are closer to the root and even the alignments may not be robust. The simplest solution to this difficulty is to narrow the analysis to subfamilies within which sequence similarity is higher. If a trace over the full family is still desired, it may then be possible to align subfamilies following a similar method.

Second, the tree establishes a natural hierarchy among class-specific residues that reflects the relative impact of their variations during evolution. The hierarchy is derived by computing successive traces as the protein family is progressively divided into more classes, defined by the branches of the tree. Thus, the first trace is computed with the entire family in one group. The second trace is done with the family divided into two classes defined by the first two branches in the tree. The third trace is done with the family divided into the three groups defined by the first three branches in the tree. This is repeated up until the family is divided into N classes, where N is the total number of sequences. In this process, every residue eventually becomes class specific, but some do so when the family is divided into fewer branches than others. By definition, a residue's evolutionary rank (rank for short) is the minimum number of branches into which it is necessary to divide the family for this residue to become class specific. Thus a residue of rank k is variable within one of the first k—1 branches of the tree, but it is invariant in each of the first k branches. Since nodes nearer to the tree root reflect the most profound evolutionary splits, residues ranked low are correlated with the most fundamental features of the protein's function. As the evolutionary rank number grows, class specificity is associated with evolutionary divergences of less and less significance, until at some rank threshold they become so trivial that class specificity loses significance. That threshold is identifiable because at that point class specific residues start to map randomly onto the surface.

Third, ET's use of the evolutionary tree follows a strategy that is closer to experimental mutational analyses than to typical computational methods. The latter are based on reasoning by analogy, that is, the analysis of protein X depends on recognizing that it bears sequence motifs also found in, say, protein A, and therefore X has some of the properties of A. Mutational analysis uses a different paradigm. It constructs variants X′, X″, X′″, and assays whether they are functionally different from X. This creates a causal link between residues and function. ET also links sequence variations with functional differences, using evolutionary divergence, or lack thereof, as its “virtual” functional assay.

If it is accepted that tree branch points are virtual assays and that ET performs retrospective mutational analysis based on evolutionary experiments, then the fourth consequence of tree usage is that ET will in fact often benefit from more mutations and more assays than are typically available in the laboratory. First, this is because the comparison of sequence yields a large number of pairwise variations (ET's equivalent of mutations). Second, and this is a crucial, because a tree with N proteins has N—1 branch points. Since each branch point is equivalent to a virtual assay, even if only a third to a half of these are above the noise threshold, (N—1)/3 is far more than the handful of assays typically available in the laboratory.

C. Gap Tolerance

In specific embodiments of the present invention, gap tolerance is included in the ET method. The inclusion of gap tolerant trace refers to a trace in which gaps are a virtual twenty-first amino acid type and/or a virtual nucleic acid. One skilled in the art realizes that this convention of interpreting a gap in the same way as any other residue does not necessarily carry biophysical meaning.

It is envisioned that gap tolerance is a computational device that is reasonable because gaps often occur in blocks in a multiple sequence alignment. These blocks typically indicate that a deletion or insertion took place that was then conserved in all descendants, suggesting some functional importance at the location of those gaps. Thus, the present invention provides the ability to rank gapped positions and eliminate holes from ET analyses and maximizes coverage of all residues in the structure.

D. Clustering Statistics

The present invention quantitates the formation of clusters to impart a quantitative parameter to the ET method, thus quantitative ET or QET. Cluster formation can be quantified using any known quantitative methods, for example clustering statistics as used herein. Thus, it is envisioned that the scope of the present invention includes any known method of determining cluster formation that is known and used by those of skill in the art.

In specific embodiments, clustering statistics are used to determine cluster formation. Specifically, statistics are employed independently or in combination with other known quantitative methods to determine cluster formation. It is further envisioned that other statistics are used in combination with the clustering statistics of the present invention.

Clusters are calculated at each rank, k, by mapping onto the structure of all trace residues with k, k−1, k−2, . . . , 2, 1 and counting the number of clusters that are formed and determining the size of the largest cluster at rank k. These numbers are compared to the expected values if the same number of the trace residues as there are at rank k had been drawn at random. The expected values can be obtained as in Example 8, when the number of trace residues at rank k is equivalent to a coverage of 0.3%, 1%, 5%, 10%, 15%, 20%, 25%, or 30%. More generally, the expected value can be generated by randomly drawing the same number of residues as there are at rank k a large number of times (typically 5000 times), and each time counting the observed number of clusters and size of the largest one. This process generates two distributions, one of the expected number of cluster, and one of the expected size of the largest one. The actual number of cluster observed and size of the largest one generated by using QET can then be compared to these distribution to evaluate the p-value of either one (Yao et al., in press). Typically a trace is deemed significant if either of these p values is ≦0.05. The user can adjust this significance threshold as appropriate, since p-values of 0.10, 0.15, and even larger are still better than random chance and may be useful to guide many of the applications of QET.

The clustering statistics comprises the overall number of clusters and/or the size of the largest cluster. One skilled in the art realizes that the number of clusters are calculated at a variety of threshold values, for example, but not limited to 0.3%, 1%, 5%, 10%, 15%, 20%, 25%, 30% and any values contained therein. Yet further, the size of the largest cluster is recorded and the distribution is plotted for each coverage level.

E. Cluster-Based Overlap Statistics

Yet further, to test the ability of trace clusters (trace residues that form significant structural clusters as described previously in the present application) to accurately predict functional sites, cluster-based overlap statistics may be used in the present invention.

The “Total Connected Residues” statistic is the total number of trace residues in the union of all clusters that overlap the functional site. The “Largest Cluster Overlap” statistic is the number of residues in the intersection of the functional site present and its largest overlapping trace cluster. The “Average Overlap” statistic is the average number of residues in overlaps between trace clusters and the functional site. Another statistic is the “Hypergeometric Distribution”, which is a non-cluster based measure of the likelihood that t out of k trace residues will overlap by chance a functional site of R residues in a protein with N residues. The p-value of t is 1-Pr(X≦t−1), where the probability mass function Pr(X) is [C(R,X)*C(N-R, k-X)/C(N, k)], and where C (x, y) denotes the binomial coefficient (the number of combinations of x objects chosen y at a time). For all statistics, the significance threshold was set at a p-value ≦0.05.

II. Implications for Large-Scale Use of the Quantitative Evolutionary Trace

In specific embodiments of the present invention, it is envisioned that the QET method is used to align remote homologs and to determine ligand binding pockets. The ability to objectively assess cluster significance, and the diminished requirement for removing gapped sequence beyond those that are obviously fragments allows for the present invention to streamline and automate QET. Thus, one skilled in the art recognizes that the present invention provides a general and natural mechanism to extract from the raw data in sequence and structure databases the answers to at least two critical biological questions: where are the functional sites, and what are their key residues?

One such embodiment is a method of aligning remote protein homologs comprising the steps of: obtaining protein sequences of at least two proteins with no sequence homology; producing a separate evolutionary trace sequence of each protein, wherein the evolutionary trace sequence identifies residues that are trace residues; assigning evolutionary rank to trace residues from each evolutionary trace; assigning an order based on the evolutionary rank; determining a correlation between any two trace residues, wherein a correlation of greater than zero indicates that the trace residues have evolutionary ranks that are dependent on each other and a correlation of zero indicates that the trace residues have evolutionary ranks that are independent of each other; aligning the evolutionary traces from the protein sequence, wherein aligning is performed to maximize the evolutionary rank order correlation from each trace; and determining a correlation between the two proteins with no sequence homology.

Another embodiment of the present invention is a method of determining a subfamily specific functional site using quantitative evolutionary trace analysis. QET is used to determine global functional determinates of a family of proteins, wherein the determinates are involved in a specific function, for example, ligand binding. Thus, the method comprises obtaining protein sequences of a subfamily of proteins within the family having a common function; aligning the protein sequences of the subfamily of proteins to generate a multiple sequence alignment; producing an evolutionary trace, wherein the evolutionary trace identifies residue that are trace residues; and comparing the evolutionary trace of the family to the evolutionary trace of the subfamily, wherein a difference in the comparison yields the functional site of the protein.

A specific embodiment is a method of determining a ligand binding pocket in a protein comprising the steps of: determining global functional determinates of a family of proteins using quantitative evolutionary trace analysis, wherein determinates are residues that are involved in the global function of the protein; obtaining protein sequences of a subfamily of proteins within the family having a common function; aligning the protein sequences of the subfamily of proteins to generate a multiple sequence alignment; producing an evolutionary trace, wherein the evolutionary trace identifies residue that are trace residues; and comparing the evolutionary trace of the family to the evolutionary trace of the subfamily, wherein a difference in the comparison yields the ligand binding pockets of the protein.

The widespread identification of evolutionarily privileged clusters of residues that are non-random and that overlap with ligand interaction sites in such a general and randomly-selected test set suggest that most proteins are amenable to trace analysis provided enough sequences are available in their respective family.

It is envisioned that proteins from a broad cross-section of structural, functional, and evolutionary characteristics are used in the present invention to determine ligand interaction, active sites or use to develop pharmaceuticals. Exemplary proteins include, but are not limited to those that participate in metabolic, signaling, transcriptional, and many other pathways where they perform catalysis, proteolysis, phosphorylation, and many other diverse biochemical activities. One skilled in the art is aware that the structures of proteins also vary widely from all α-helix, all β-sheet, α-helix and β-sheet containing proteins, and one integral membrane protein (visual rhodopsin).

The proteins are isolated from a range of species, including eukaryotic (mammals, plants, fungi, and others), prokaryotic (Eubacteria & Archaebacteria), and viral representatives. Eukaryotic examples include, but are not limited to HSP-90 and growth hormone receptor; prokaryotic examples include β-Lactamase and citrate synthase; and viral examples include, but are not limited to HIV reverse transcriptase and F-MuLV.

III. Applications of Quantitative Evolutionary Trace

It is envisioned that the present invention has practical applications for rational drug development and protein engineering. Yet further, it is contemplated that the present invention provides the means of integrating sequence and structure databases to extract information on the molecular basis of function.

A. Rational Drug Design

In specific embodiments of the present invention, QET is used in the designing of pharmaceuticals that target a protein and/or nucleic acid. In one such method, it is envisioned that the design of a pharmaceutical comprises the steps of obtaining a protein sequence; aligning the protein sequence to homologous protein sequences to generate a multiple sequence alignment; predicting at least one residue in the protein sequence which is involved in the protein's function, wherein predicting the residues involves using quantitative evolutionary trace analysis; and synthesizing the pharmaceutical to interact with the predicted residue in the protein. One skilled in the art is cognizant that the pharmaceutical is a protein, peptide, small molecule, nucleic acid or a combination thereof.

The goal of rational drug design is to produce structural analogs of biologically active compounds. By creating such analogs, it is possible to fashion drugs, which are more active or stable than the natural molecules, which have different susceptibility to alteration or which may affect the function of various other molecules. In one approach, one would generate a three-dimensional structure for the protein of interest. This could be accomplished by X-ray crystallography, computer modeling or by a combination of both approaches. An alternative approach involves the random replacement of functional groups throughout the protein and the resulting affect on function determined.

It also is possible to isolate a specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have enhanced and improved biological activity, for example, for the target of interest. In addition, knowledge of the chemical characteristics of these compounds permits computer employed predictions of structure-function relationships.

B. Protein Engineering

The present invention can be used to design proteins, protein engineering, that have desired and/or altered protein properties. For example, the method may comprise the steps of: obtaining a protein sequence; aligning the protein sequence to homologous protein sequences to generate a multiple sequence alignment; predicting at least one residue in the protein sequence which is involved in the protein's functions, wherein predicting the residues involves using quantitative evolutionary trace analysis; synthesizing libraries of protein variants wherein residues at and/or around the predicted functional site are substituted with alternative amino acids; and screening the resulting libraries for mutant proteins with the desired protein properties.

Protein properties include, for example, but are not limited to binding affinity, aggregation, crystallization, solubility, stability (e.g., degradation, post-translational modification), immunogenicity, and other properties that are part of the protein's normal biological, cellular, and physico-chemical behavior and effects. It is envisioned that the mutated protein possess at least one of these protein properties. Yet further, it is envisioned that the residues that are mutated result in an enhanced and/or decreased desired protein property.

Yet further, another embodiment is a method to identify residues on a protein structure that are least likely to be part of a functional site that is critical to biochemical activity, binding, or structure folding and stability. The residues are identified using quantitative evolutionary trace analysis. Such residues can be targeted for mutation to impart altered protein properties to the protein with little risk of destroying the protein's fold, structure, and normal function. Thus, it is envisioned that the mutations can result in a variety of altered protein properties (e.g., increase and/or decrease), for example, but not limited to binding affinity, aggregation, crystallization, solubility, stability (e.g., degradation, post-translational modification), immunogenicity, and other properties that are part of the protein's normal biological, cellular, and physico-chemical behavior and effects. Yet further, depending upon the desired protein property, the residues can be mutated to enhance and/or decrease any of the desired protein properties.

1. Binding Affinity

Binding affinity is the measure of the overall free energy of the interaction between the protein and a ligand. The magnitude of the affinity determines whether a particular interaction is relevant under a given set of conditions. Whether or not any particular affinity of a protein for a ligand is significant depends on the concentration of the ligand present for the protein to encounter. Assays for determining binding affinity include, but are not limited to, surface plasmon resonance, Western blot, ELISA, DNase footprinting, and gel mobility shift assays. The ligand may be protein or non-protein. The ligand may be, but is not limited to, a receptor, a coenzyme, or a non-proteinaceous chemical compound. Binding affinity between a protein and ligand may be measured by the association or dissociation constant of the binding between the protein and the ligand. Entropy of binding between the protein and ligand may be decreased by stabilizing structures similar to that of the protein in a bound state with the ligand. van der Waals calculations can be performed with the protein and the ligand to determine whether binding conformation will be sterically allowed.

2. Aggregation of Proteins

Protein aggregation refers to the interaction of proteins, usually non-specific, to form a complex that may or may not be covalently linked. Aggregation can occur as a competing reaction to folding. Aggregation often causes irreversible precipitation and in vivo can lead to degradation of the complex. Aggregates may form due to exposed hydrophobic areas on partially folded proteins. This may occur with any exposed hydrophobic region, even in a folded protein. Aggregation is a problem in the production of recombinant proteins. This is troublesome in the production of peptides and proteins for pharmaceutical use. Aggregation of proteins or peptides in solution can be determined by measuring light scattering at 360 nanometers as well as by analytical centrifugation. Glutamine/asparagine amino acid rich domains within a protein have been shown to predispose a protein to aggregation.

3. Protein Crystallization

Crystallization is one of several means (including nonspecific aggregation/precipitation) by which a metastable supersaturated solution reaches a stable lower energy state by reduction of solute concentration. It is a pre-requisite for structure determination by X-ray crystallography.

Chemical/biochemical modification of proteins may be used to change crystallization conditions. Electrostatic surface characteristics play a large role in dictating whether a protein crystallizes or not. Thus, modification of surface charges by either chemical (derivitization) or biochemical (mutagenesis) means can provide crystals.

For example, biochemical modification in the form of site directed mutagenesis of surface residues has been utilized to improve the crystallization characteristics of human thymidylate synthase (McElroy et al., 1992). The protein initially crystallized in such a way as to make it impossible to interpret the active site in electron density maps due to disorder.

In specific embodiments, single point mutations at non-conserved surface residues may be designed either to neutralize charges (mutation to asparagine), reverse charges (arginine and lysine to glutamate or aspartate, and vice-versa), or add charges (cysteine, proline, leucine, and glutamine to aspartate, glutamate, or lysine).

4. Solubility of Proteins

The solubility of a protein is the amount of the protein that can be dissolved in a given volume of a solvent. The presence of greater than this amount of the protein will cause the protein to aggregate and precipitate. The solubility of a protein in water is determined by its free energy when surrounded by aqueous solvent relative to its free energy when interacting in an amorphous or ordered solid state with any other molecules that might be present, or when immersed in membranes. A factor in the solubility of any substance is the amount of energy required to displace the buffer to accommodate the substance. Ionic strength, pH and temperature of the buffer affect the solubility of a protein. Increasing the ionic strength of the buffer at low values tends to increase solubility of the protein, while increasing ionic strength at high values tends to decrease solubility. In a low ionic strength buffer, the protein is surrounded by an excess of ions of charge opposite to the net charge of the protein. This decreases the electrostatic free energy of the protein and increases solubility. In an aqueous solvent, charged and polar groups on the surface of the protein interact favorably with water. Organic solvents tend to decrease the solubility of proteins. A protein is least soluble at its isoelectric point. At a pH above the isoelectric point, the protein is deprotonated and soluble. At a pH below the isoelectric point, the protein is protonated and soluble. The greater the net charge on a protein, the more likely they are to stay in solution. This is due to the greater electrostatic repulsions between molecules. High temperature causes proteins to denature, thus aggregating and losing solubility. High solubility is a requirement for structure determination by Nuclear Magnetic Resonance spectroscopy.

5. Immunogenicity of Proteins

The immunogenicity of a protein is based upon it binding to proteins of the major histocompatibility complex (MHC). Factors which decrease the likelihood of that occurrence decrease immunogenicity. MHC molecules present the antigen to antibodies. T cells recognize peptide/MHC complexes in the adaptive immune response to antigens. A protein pharmaceutical that is bound by the MHC will not arrive at its site of effectiveness, nor will future molecules of the protein pharmaceutical. Therefore, it is a key objective to design protein pharmaceuticals with low immunogenicity. A smaller protein is less likely to be recognized by the MHC. Therefore, aggregates of a protein can cause increased immunogenicity. In addition, aggregates can trigger degradation which will allow recognition of parts of the protein which are normally inaccessible within the folded protein. Therefore an increase in the stability of a protein will aid in decreasing immunogenicity.

6. Protein Stability

One potential way of increasing the stability of a protein is to introduce new disulfide bonds into the protein. A disulfide bond may stabilize the folded state of the protein relative to its unfolded state. The disulfide bond accomplishes such a stabilization by holding together the two cysteine residues in close proximity. Without the disulfide bond, these residues would be in close proximity in the unfolded state only a small fraction of the time. This restriction of the conformational entropy (disorder) of the unfolded state destabilizes the unfolded state and thus shifts the equilibrium to favor the folded state. The effect of the disulfide bond on the folded state is more difficult to predict. It could increase, decrease or have no effect on the free energy of the folded state. Increasing the free energy of the folded state may lead to a destabilization of the protein, which would tend to cause unfolding. Importantly, the cysteine residues which participate in a disulfide bond need not be located near to one another in a protein's primary amino acid sequence.

Another potential way of increasing the stability of a protein is stabilizing the N-terminal amino acid of the protein. For example, in bacteria, long-live proteins have a chemically modified (“blocked”) N-terminus. The most frequent modification is acetylation, which prevents the N-terminal amino acid from being degraded.

Yet further, another potential way of increasing the stability of a protein is to ensure that the protein is correctly folded or assembled. Missassembled or misfolded proteins are targeted for degradation by the cell's degradation pathways, for example, but not limited to ubiquitin-dependent proteolytic pathway, the endoplasmic reticulum or proteasome or lysosome. Thus, a mutation resulting in the proper folding or assembly of a protein will prevent degradation by the cell's normal processes.

B. Mutagenesis

It is also envisioned that the present invention is used to assess the effects of point mutations or other mutations that result in an change in the functional site of the structure or result in a change in a given protein property. Thus, QET can be used to design pharmaceuticals that are agonist, antagonists or inverse agonists or have altered protein properties and/or functions, which has been discussed previously in this application.

Thus, where employed, mutagenesis is accomplished by a variety of standard, mutagenic procedures. Mutation is the process whereby changes occur in the quantity or structure of an organism. Changes may be the consequence of point mutations that involve the removal, addition or substitution of a single nucleotide base within a DNA sequence, or they may be the consequence of changes involving the insertion or deletion of large numbers of nucleotides or insertion, deletion and/or substitution of amino acids in the protein and/or peptide sequence.

Structure-guided site-specific mutagenesis represents a powerful tool for the dissection and engineering of protein interactions. The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Other methods of site-directed mutagenesis are disclosed in U.S. Pat. Nos. 5,220,007; 5,284,760; 5,354,670; 5,366,878; 5,389,514; 5,635,377; and 5,789,166.

C. Mimetics

The present inventors also contemplates that structurally similar compounds may be formulated to mimic the key portions of protein or peptides that are determined by the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al., 1998). A structural motif that appears to be evolutionarily conserved in certain toxins is small (30-40 amino acids), stable, and high permissive for mutation. This motif is composed of a beta sheet and an alpha helix bridged in the interior core by three disulfides.

Beta II turns have been mimicked successfully using cyclic L-pentapeptides and those with D-amino acids (Weisshoff et al., 1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

D. Assessment of SNP

In further embodiments, it is envisioned that the ET method is used to assess the significance of a single nucleotide polymorphism (SNP) that is located in or near a functional site.

The method of determining the significance of a single nucleotide polymorphism in a protein, wherein the single nucleotide polymorphism occurs in a predicted trace residue comprises the steps of: performing a quantitative evolutionary trace analysis on a protein; performing a quantitative evolutionary trace analysis on a protein suspected of containing a single nucleotide polymorphism; comparing the analysis on the protein to the protein suspected of containing a single nucleotide polymorphism; and assessing whether if the single nucleotide polymorphism occurs in a residue that is predicted to be a functional site of the protein.

If the rank of the affected residue is statistically significant, i.e., at a level where clustering is significant, then the SNP is suggested to be functionally important. Yet further, if the affected residue falls directly in the statistically significant and largest cluster, then the SNP is functionally important.

Thus, the present invention can be used to identify plausible disease candidates among SNPs that cause mutations in or near the functional sites, i.e., missense substitutions.

E. Biological Functional Equivalents

In specific embodiments of the present invention mutations, modifications and/or changes are made in the structure of the proteins. QET can be used to predict residues that are essential to protein function or predict residues that are not required for protein function, but if altered can play a role in protein function. Thus, based upon the predicted residues, biological functional equivalents can be generated as another means to develop pharmaceuticals and/or protein engineering.

1. Modified Proteins

The biological functional equivalent may comprise a protein that has been engineered to contain distinct sequences while at the same time retaining the capacity to encode the “wild-type” or standard protein. This can be accomplished to the degeneracy of the genetic code, i.e., the presence of multiple codons, which encode for the same amino acids. In one example, one of skill in the art may wish to introduce a restriction enzyme recognition sequence into a protein while not disturbing the ability of that polynucleotide to encode a protein.

In another example, a polynucleotide can be (and encode) a biological functional equivalent with more significant changes. Certain amino acids may be substituted for other amino acids in a protein structure without appreciable loss of interactive binding capacity with structures such as, for example, antigen-binding regions of antibodies, binding sites on substrate molecules, receptors, and such like. So-called conservative changes do not disrupt the biological activity of the protein, as the change is not one that impinges of the protein's ability to carry out its designed function. It is thus contemplated by the inventors that various changes may be made in the sequence of genes and proteins disclosed herein, while still fulfilling the goals of the present invention.

In terms of functional equivalents, it is well understood by the skilled artisan that, inherent in the definition of a biologically functional equivalent protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) in selected amino acids (or codons) that can be substituted.

In general, the shorter the length of the molecule, the fewer changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and/or arginine (−4.5).

The importance of the hydropathic amino acid index in conferring interactive biological function on a protein is generally understood in the art. It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±0.5 are even more particularly preferred.

It also is understood in the art that the substitution of like amino acids can be made effectively on the basis of hydrophilicity, particularly where the biological functional equivalent protein and/or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and/or those within ±0.5 are even more particularly preferred.

2. Altered Amino Acids

The present invention, in many aspects, relies on the synthesis of proteins and polypeptides in cyto, via transcription and translation of appropriate polynucleotides. These proteins and polypeptides will include the twenty “natural” amino acids, and post-translational modifications thereof. However, in vitro peptide synthesis permits the use of modified and/or unusual amino acids. A table of exemplary, but not limiting, modified and/or unusual amino acids is provided herein below.

TABLE 1
Modified and/or Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, beta- | Ahyl | allo-Hydroxylysine |
| | Amino-propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic | Orn | Ornithine |
| | acid | | |
| EtGly | N-Ethylglycine | | |

IV. Screening Assays

It is envisioned that the protein and/or peptide databases that are generated using the present invention are used to screen other libraries and/or databases for molecules that target the databases of the present invention.

An exemplary screening method includes, but is not limited to a method of screening compounds comprising the steps of: obtaining a protein having predicted functional sites, wherein the functional sites are predicted using quantitative evolutionary trace analysis; contacting the protein with a candidate substance; determining whether the candidate substance interacts with the protein, wherein interaction with the protein indicates that the candidate substance is a ligand.

A variety of assays can be used in the present invention to determine if the candidate substance is a ligand. For example, a quick, inexpensive and easy assay to run is an in vitro assay. Various cell lines can be utilized for such screening assays, including cells specifically engineered for this purpose. Depending on the assay, culture may be required. Alternatively, molecular analysis may be performed, for example, looking at protein expression, mRNA expression (including differential display of whole cell or polyA RNA) and others.

In addition to in vitro assays, in vivo assays involve the use of various animal models, including transgenic animals. Due to their size, ease of handling, and information on their physiology and genetic make-up, mice are a preferred embodiment, especially for transgenics. However, other animals are suitable as well, including insects, nematodes, rats, rabbits, hamsters, guinea pigs, gerbils, woodchucks, cats, dogs, sheep, goats, pigs, cows, horses and monkeys (including chimps, gibbons and baboons). Assays of protein pharmaceuticals may be conducted using an animal model derived from any of these species or others.

In such assays, one or more candidate substances are administered to an animal, and the activity of the candidate substance(s) as compared to a similar animal not treated with the candidate substance(s) is measured.

Treatment of these animals with candidate substances will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

### V. EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Determination of Trace Residues in SH2and SH3Domains

Evolutionary trace analysis was used to determine the class-specific residues in SH2 and SH3 modular signaling domains.

Briefly, the first step in the evolutionary trace (ET) was to align all relevant amino acid sequences from which a sequence identity tree, or dendrogram, was attained (FIG. 1). Next, the tree was divided into groups, and the invariant residues in each group defined its consensus sequence. Third, consensus sequences were compared, producing a trace sequence. Residue positions that had conserved residues within each group, but among the groups had different identities were called class specific (for example, positions 1, 2 and 11). Positions that had completely conserved amino acids across all family members were called invariant (position 1 has rank 3). These trace residues, both class specific and invariant, were finally mapped onto a representative three-dimensional structure. If these residue cluster on the structure, then this site was considered to be of evolutionary importance and was likely an active site on the protein.

In SH2 and SH3 modular signaling domains, and in the zinc finger domain of intracellular hormone receptors, the optimally ranked class-specific residues formed a tight cluster on the protein structure while the remaining protein surface was free of ET signal (Lichtarge et al., 1996; Lichtarge et al., 1997). These clusters matched the interfaces known from structures of the protein-ligand complex.

In the SH3 analysis, a discrepancy between the predicted and the observed structural interface reproduced that found by alanine scanning mutagenesis. Thus, the evolutionary mutational experiments embodied in ET analysis matched those from the laboratory and added functional insights to the protein structure.

### Example 2

### Mutations in SH2 Domains and Zinc Finger Domains

Comparison with mutational studies further suggested that evolutionary rank was linked to functional specificity.

One example was SH2 domain mutations. Mutations at low-rank residues eradicated activity. Mutations of higher rank residues modulated activity without destroying it outright. Mutations of the highest ranked residues had no effect on function.

Another example was in the zinc finger domain of nuclear hormone receptors where low-ranked class-specific residues fell into two qualitatively different groups. The first group appeared to define the essential characteristic of DNA binding. It contained residues with ranks between 1 and 7 that either were invariant or underwent at most one substitution and contact the most conserved bases of the DNA response element. In contrast, the second group appeared to define the specific response element that was recognized. It consisted of positions with ranks between 8 and 21 that underwent many and highly nonconservative substitutions, and which contacted DNA bases that were themselves so variable as to fall outside the consensus sequence.

These examples showed that some active sites through variations on a conserved structural framework and that sequence identity trees yielded reasonable approximations of functional classifications.

### Example 3

### Evolutionary Traces Along G Protein Signaling Pathway: Active Sites in Gα Subunits

In the Gα family, the evolutionary trace identified a likely receptor interface whose role was confirmed by mutational analysis. These and other studies severely constrained the receptor—G protein interaction and led to a model of the quaternary structure of the GPCR-G protein complex.

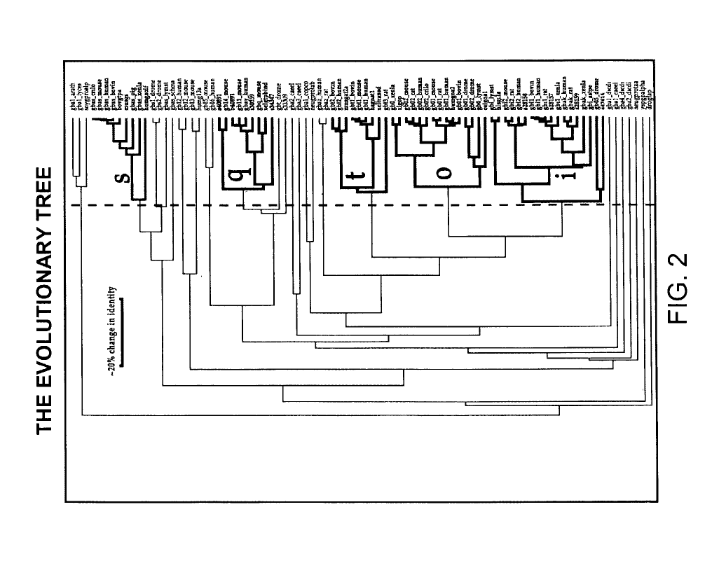

Briefly, sequences of 88 G-protein α-subunits were retrieved from SwissProt and aligned using the GCG program PILEUP. The resulting dendrogram is shown in FIG. 2. The dashed line indicated the tree partition where the five principal G α subgroups were separated. Since functional subgroups were attained from appropriate division of the dendrogram, identity trees were used to approximate a functional classification of the members of a protein family.

The ET analysis yielded three clusters of class-specific residues. One was at the nucleotide binding cleft, and the other two formed distinct functional surfaces on opposite sides of the Gα Ras-like domain. The first of these two surfaces, A1, comprised 17 residues from the distal two-thirds of helix α₅, the sixth β-strand (β₆), the α₄/β₆ loop, the N-terminal ends of β₄ and β₅ and the C-terminal tail, following the nomenclature of Noel et al. The second surface, A2, was larger with 32 residues distributed on either side of helix α₂ and strands β₁, β₂, helix β₃, and loops β₃/α₂, β₄/α₃, and β₁/α₂. On the strength of data linking the C-terminal tail to receptor specificity, site A1 was initially thought to be the receptor interface leaving A2 as the logical interface with G β. The Gαβγ trimer structure shows that indeed the Gα-Gβ interface spans nearly two-thirds of A2.

The role of site A1 in receptor coupling was further confirmed by constructing 100 Gᵢα mutants with either single (92) or double (8) replacement of residues with alanine (Onrust et al., 1997). Each mutant was classified either as wild-type or as having impaired coupling to the receptor based on two assays: (1) a decrease in the rate of Gᵢα hydrolysis by trypsin, or (2) a decrease in Gᵢα binding to photoactivated rhodopsin (the receptor responsible for the physiological activation of transducin).

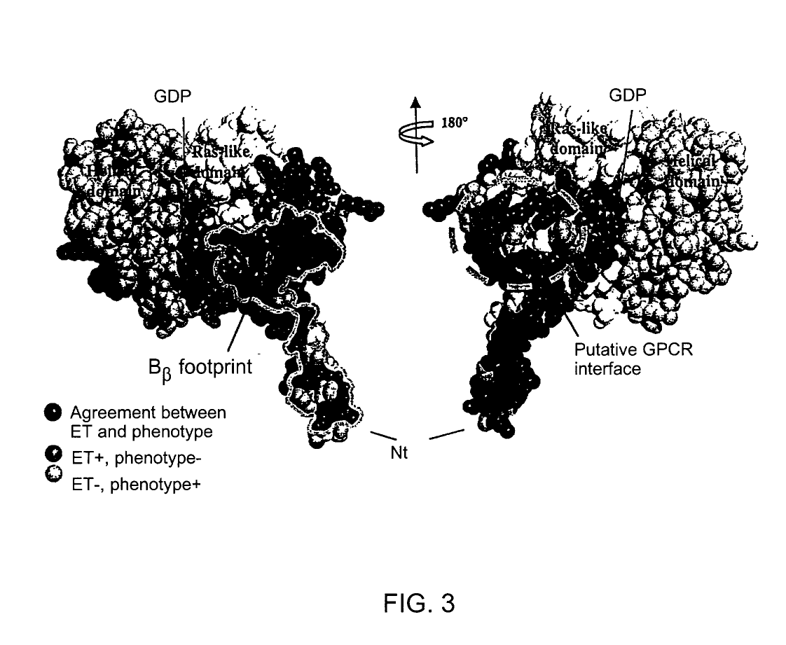

FIG. 3 displays, on both sides of the Gᵢα structure, the comparison between the predicted importance of residues and the effect that alanine substitutions has on GPCR coupling. The overall agreement, shown in black, is 68% (sensitivity, 75%; specificity 65%; p=0.002) indicated that nearly 7 out of 10 times ET analysis correctly anticipated whether an alanine would or would not alter function.

This outcome may underestimate ET's predictive accuracy, however, because many of the residues that were predicted to be important, but yet produced no functional change upon alanine substitution, shown in light gray, were located at the nucleotide binding cleft or at the Gαβγ binding site. Therefore, it was likely that some of these residues were important for signaling, but not as measured by assays aiming to detect GPCR interaction. At site A1, where the experimental assay and ET analysis were most likely to measure the same effect, the positive predictive value of ET rises to 79%. Thus, this discrepancy highlights an important difference between mutational analysis based on evolution and that based on the laboratory experiments: the former is sensitive to all the functions of a protein, whereas the latter focuses on the specific properties tested for by the assays.

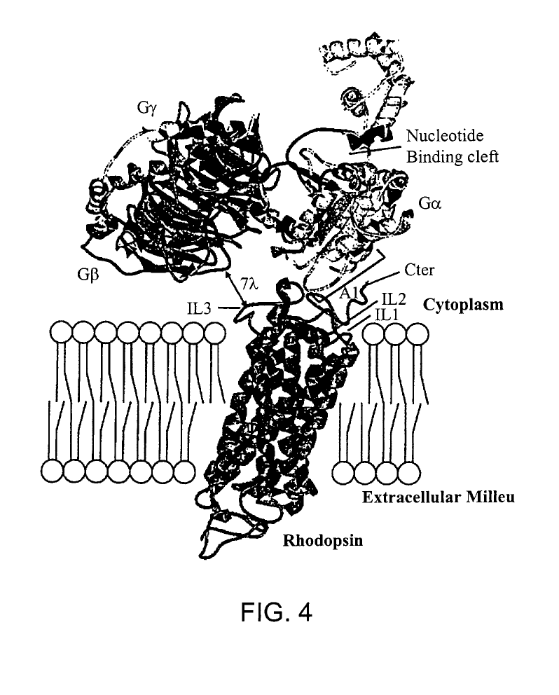

Yet further, the agreement between evolutionary and laboratory-based mutational analysis strongly suggested that A1 is an interface to the G-protein-coupled receptor (GPCR), and this knowledge was incorporated into a model of the GPCR-G protein complex shown in FIG. 4. This model was also based on the lipid modifications of the N terminus of Gα and the C terminus of Gγ, which suggested that both interact with the membrane; on contacts between the third intracellular loop of the receptor with C terminus (Kostenis et al., 1988) and the N terminus of Gα, (Taylor et al., 1994) and also with Gβγ (Wu et al., 1998); and between the fourth intracellular loop and Gαβγ (Phillips and Cerione, 1992). This configuration created a favorable charge interaction between the bilayer and the G αβγ complex, (Lambright et al., 1996) and it showed that the short intracytoplasmic loops of the receptor cannot reach the nucleotide binding cleft. Thus, exchange of GDP for GTP was triggered “at a distance” through an allosteric pathway. Pending a definitive structure of a GPCR-G protein complex, this model also provided a useful context in which to discuss experimental studies of their interactions. In particular, it was interesting that intracellular loop was the most likely contact between the receptor and site A1, while intracellular loop 3 was wedged between Gα and G βγ, where it was accessible to the N- and C-terminals of G α and to G βγ. The fourth intracellular loop of the receptor interacted with the G βγ and the first intracellular loop was farthest from the G protein, consistent with the paucity of any data on its role in G protein coupling.

### Example 4

### Evolutionary Traces Along G Protein Signaling Pathway: Active Sites in RGS Proteins

Downstream from the receptor, the G α-GTP complex activates effectors, enzymes, and ion channels, until it reverts to its inactive G α-GDP state. The intrinsic rate of GTP hydrolysis by G α is too slow, however, to account for the rate at which signaling is turned off. The regulators of G protein signaling (RGS) proteins (Watson et al., 1996; Berman et al., 1996; Berman and Gilman, 1998; Druey et al., 1996) reconcile this difference by binding to and stabilizing the Gα catalytic switch regions, (Tesmer et al., 1997) increasing the rate of Gα-GTP hydrolysis. The existence of RGS proteins in general and the diversity of family members indicated that regulation of RGS proteins may add yet another level of control in G protein signaling. For example, the inactivation of Gᵢα (the G protein of vision) via RGS9 (the physiological GAP for Gᵢα) is enhanced in the presence of the γ subunit of the cGMP phosphodiesterase (the effector which Gᵢα activates). However, PDE γ inhibits RGS4, RGS16, GAIP, and the RGS9 subfamily members RGS6 and RGS7. In order to understand how the RGS domain may be regulated by effectors (He et al., 1998; Arshavsky and Bownds, 1992; Angleson and Wensel, 1994) or other factors, (Benzing et al., 2000; Aheng et al., 2000; Kovoor et al, 2000), an ET analysis of 42 members of the RGS family was preformed and the results were mapped to the only RGS structure available at the time, RGS4 (Tesmer et al., 1997).

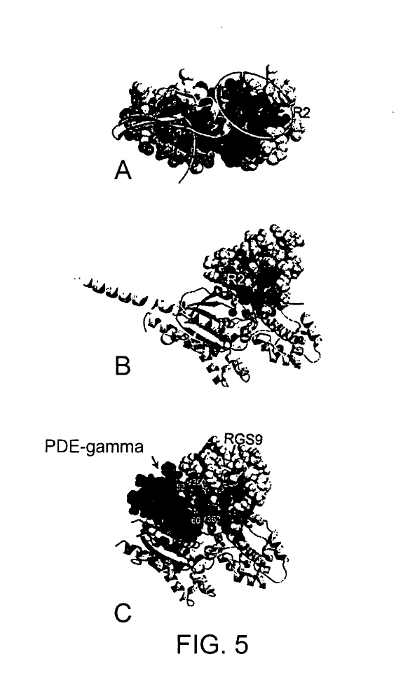

A trace of the RGS family identified a large cluster of both invariant and class-specific residues on the surface of the representative RGS4 domain (FIG. 5). These residues had ranks below 20 while the rest of the protein's surface remained free of signal until rank 23, suggesting this site is functionally important. This was consistent with the known RGS4-Gᵢⱼα GDP AIF4 structure (Tesmer et al., 1997) since 10 of the 11 RGS residues at the RGSR-Gα interface fall within this cluster. The remaining 7 residues, if taken by themselves, formed a second, smaller cluster, R2, that extended beyond the Gα-binding site and whose function was unknown a priori.

Two observations suggested that site R2 was an interface whereby the effector could influence RGS domain activity. First, amino acids in this region varied in a manner that was consistent with the unique activity of distinct RGS proteins in the presence of the PDEγ. Specifically, in proteins inhibited by PDEγ, the residues at RGS4 position 117 were acidic, and at position 124 they were either polar or hydrophobic, but these residues were hydrophobic (L) and basic (K), respectively, in RGS9 which was enhanced by PDEγ. Second, in the Gα-RGS complex, site R2 was in near contiguity with to a part of cluster A2 in Gα that (a) did not interact with Gβ and (b) contained residues linked to PDEγ interaction. Thus, in order to influence GTPase activity, the effector was likely to bind the RGS-Gα complex by spanning part of A2 and R2 (FIG. 5) (Sowa et al., 2000).

Structural data supports a role for R2 in mediating these interactions. The structure of the catalytic core domain of RGS9 in complex with both Gᵥᵢⱼα-GDP A1F4 and the C-terminal 38 amino acids of PDEγ reveals that PDEγ V66 contacts R2 at class-specific residue RGS9-W362 (RGS4-126) (FIG. 5C). A second R2 residue RGS9-R360 (RGS4-124) was in close proximity to PDEγ D52 (Slep et al., 2001). Moreover, other R2 residues in the α₅/α₆ connecting loop lie parallel to the effector binding site on Gα, suggesting that they play a role in positioning the RGS domain for interactions with both the effector and the effector bound Gα.

### Example 5

### Allostery and Specificity in RGS

Mutational analysis revealed that residues at site R2 effect the activity of the RGS domain, even though they did not directly contact Gᵢα.

Briefly, a series of mutations were made in RGS7, the protein with the most closely related RGS domain to RGS9, yet potently inhibited by PDEγ (Sowa et al., 2001). Mutations were made in the RGS7 catalytic core domain at residues in site R2, replacing these residues with their corresponding amino acids from RGS9. The proteins were then assayed for their ability to increase the rate of GTP hydrolysis by Gᵢα in either the absence or presence of PDEγ (Cowan et al., 2000). Δκ_{inact} was calculated as: Δκ_{inact}=(κ_{inact}(RGS+Gₜα)], with κ_{inact} calculated by fitting the time course of GTP hydrolysis to: % GTP hydrolyzed=100(1−exp[−κ_{inact} time]).

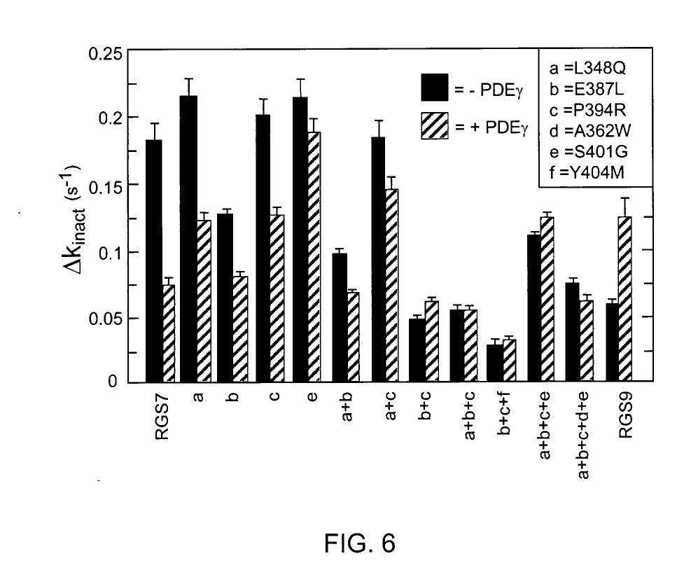

Class-specific residues in RGS7 corresponding to RGS4 position 77 (RGS7-348), 117 (RGS7-387), and 124 (RGS7-394) were mutated singly, doubly, and all together to their corresponding amino acids from RGS9. Both E387L/P394R and the triple mutants were remarkable for reduced basal activity, down to a level slightly less than the PDEγ inhibited form of the wild-type RGS7. Addition of PDEγ was shown in FIG. 6. The mutation at residue 348 had little effect by itself, and the triple mutant behaved nearly the same as the E387L/P394R mutant. Thus, class-specific residues 387 and 394 were critical for regulating RGS7 domain activity, and changes at these positions were sufficiently drastic as to alter RGS conformation or dynamics in the manner that appeared to mimic the PDEγ inhibited form of the wild-type protein.

In order to determine which residues controlled the RGS-specific enhancement or inhibition effect of PDEγ, additional mutations were made at the RGS/Gα interface. The triple mutant E387L/P394R/Y404M remained insensitive to PDEγ, but had a reduced activity as compared to E387L/P394R (FIG. 6). Mutant L348Q/E3871/P394R/S401G had basal activity that was nearly that of wild-type RGS9 with PDEγ, and on PDEγ addition was slightly enhanced to match exactly the activity of RGS9 with PDEγ. Adding the mutation A396W to produce the mutant L348Q/E387L/P394R/A396W/S401G created a protein that now was slightly inhibited in the presence of PDEγ, possibly due to structural restrictions imposed by surrounding residues (FIG. 6). Thus, residue S401 in RGS7 appeared to be a critical determinant of the direction of the PDEγ effect on Gα. However, S401 required the assistance of 387 and 294 since when S401 was mutated alone, the protein remained inhibited by PDEγ, indicating an allosteric relationship between 387/394 and 401 (Sowa et al., 2001).

A network of residues connected 387/394 to Gα contact residue 401 through the α₅/α₆-connecting loop. Residues 387 was located N-terminal to the α₅/α₆-connecting loop in which lies P394. This loop was critical for the GTPase accelerating activity of the RGS domain (Slep et al., 2001; Natochin et al., 1988) and was composed almost entirely of class-specific residues, consistent with an important role for the entire protein family. Residues at positions corresponding to 387 and 394 may exert their influence by communicating through this loop to the catalytic interface, with specificity determined by the amino acids that comprise both the α₅/α₆-connecting loop and the RGS/Gα interaction surface.

These results illustrated that the knowledge of class-specific residues helped direct mutagenesis to identify and unravel the interplay of functional elements in a multiprotein complex, including an intraprotein allosteric pathway.

### Example 6

### Signal Transduction in G-Protein-Coupeled Receptors

ET was used to identify which residues in G-protein-coupled receptors (GPCRs) mediate general signal transduction properties and were responsible for ligand-specific functions. This distinction was possible because ligands were extremely diverse in size and character, whereas G proteins were much more conserved and coupled to receptors in both a one-to-many and many-to-one fashion. It follows that ligand binding was highly specific while signal transduction and G protein coupling was likely more generic. To distinguish the functional determinants responsible for these distinct aspects of GPCR function, the approach was to identify positions that were important to all receptors and compare them to those that were important to all receptors and compare them to those that were important in a given subfamily.

In order to find the global functional determinants, GPCRs were selected broadly, including 58 opsins, 58 adrenergic receptors, 63 chemokine-related receptors, and 30 olfactory receptors, all in Class A, as well as 33 secretin-related receptors, from Class B. Before an evolutionary trace was computed on all these receptors, it was necessary to align them, which was difficult because members of Class B have no sequence homology and traditionally cannot be aligned to members of Class A.

Correlation of evolutionary ranks at cognate residues was used to identify and align remote homologs. Briefly, five receptor families were traced separately, to assign an evolutionary rank at every position of their seven transmembrane helices, TM1 to TM7. Residues were then assigned an order based on their ET rank. For example, if in opsins residue position 1 has an ET rank of 15, which was the fifth overall lowest rank in the sequence, then position 1 was assigned the evolutionary rank order of 5. When this was repeated in each receptor family, it became possible to compute the Spearman rank-order correlation between any two. A perfect correlation meant that the relative rank of cognate residues was perfectly matched between two families. A correlation of 0, however, indicated that cognate residues had evolutionary ranks that were completely independent. As might be expected, the evolutionary ranks among Class A receptors were positively correlated, presumably reflecting a common origin, but the correlations were modest and not equal in all helices, consistent with significant functional divergences (Table II).

TABLE II
SPEARMAN RANK-ORDER CORRELATION COEFFICIENTS FOR GPCRs^{a}

| | | | | | | | Average |
|---|---|---|---|---|---|---|---|
| Correlation | OP-AD | OP-TH | AD-TH | OP-OL | AD-OL | TH-OL | global |
| TM1 | 0.09 | 0.28 | 0.3 | 0.33 | 0.34 | 0.12 | 0.24 |
| TM2 | 0.07 | 0.32 | 0.32 | 0.12 | 0.45* | 0.36 | 0.27 |
| TM3 | 0.58*** | 0.40* | 0.40* | 0.45** | 0.05 | 0.49** | 0.40* |
| TM4 | 0.21 | 0.21 | 0.41* | 0.56** | 0.36 | 0.37 | 0.35 |
| TM5 | 0.56** | 0.64*** | 0.64*** | 0.41* | 0.28 | 0.21 | 0.46* |
| TM6 | 0.52** | 0.38* | 0.37* | 0.04 | 0.22 | −0.38 | 0.19 |
| TM7 | 0.41* | 0.54** | 0.33 | 0.19 | 0.65*** | 0.34 | 0.41* |
| Overall | 0.43*** | 0.43*** | 0.49*** | 0.35*** | 0.39*** | 0.33*** | 0.40*** |
| ^{a}The Spearman rank-order correlation coefficients are shown for the indicated comparisons of GPCR classes (OP, opsins; AD, adrenergic; TH, chemokine-related; OL, olfactory; TM, transmembrane helix; *, p < 0.05; **, p < 0.01; ***, p < 0.001). The average global correlation is the average of all comparisons for a given helix. The overall correlation was determined by first concatenating the ET results for all 7 helices within each group and then calculating | | | | | | | |
| # the correlation between the indicated groups. | | | | | | | |

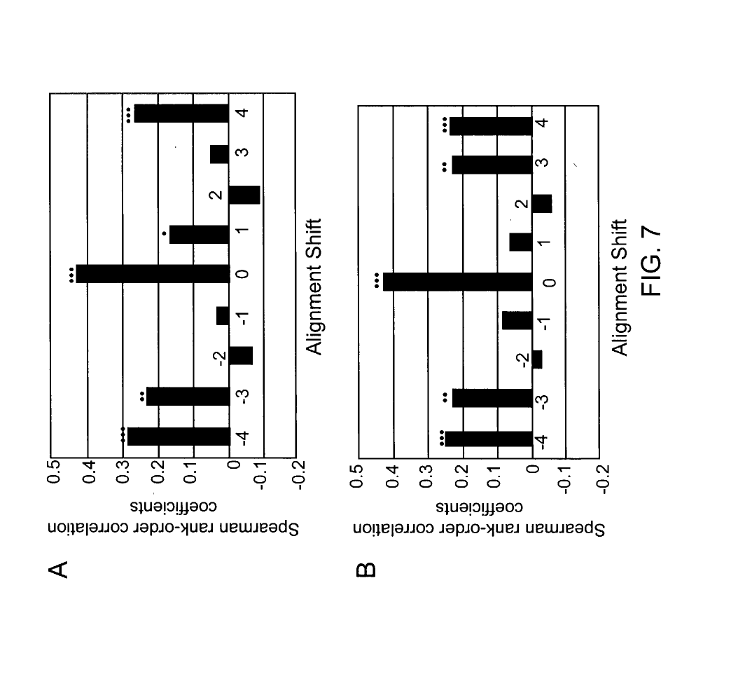

Importantly, rank-order correlation was a sensitive indicator that two groups were correctly aligned. This was shown in FIG. 7, where the misalignment of visual and adrenergic receptors by up to ±4 positions was associated with a decrease in their correlation. The minimum was at ±2, because low-ranked residues were mostly internal. Hence, when the internal residues of one helix (low-ranked) were compared to lipid-facing (high-ranked) residues in the other, ±2, the correlation was least. Interestingly, even when the helices were back in phase at ±4, the correlation did not fully recover. Thus, the amphipathic nature of low versus high ranked residues was not sufficient alone to yield the maximum correlation of evolutionary ranks. The difference, although small, should reflect the cognate residues involved in similar functions have an additional degree of rank correlation.

TABLE III

ALIGNMENT BETWEEN BOVINE VISUAL RHODOPSIN AND
HUMAN PARATHYROID HORMONE RECEPTOR

| Corre- | | |
|---|---|---|
| lation | Source | Sequence |
| TM1 | OPS: | QFSMLAAYMFLLIMLGFPINFLTLYVTVQ |
| | PTH: | VFDRLGMIYTVGYSVSLASLTVAVLILAY |
| TM2 | OPS | LNYILLNLAVADLFMVFGGFTTTLYT |
| | PTH: | RNYQHMHLFLSFMLRAVSIIFVKDAVL |
| TM3 | OPS | TGCNLEGFFATLGGEIALWSLVVLAIERYVVVCK |
| | PTH: | AGCRVAVTFFLYFLATNYYWILVEGLYLHSLIFM |
| TM4 | OPS | AIMGVAFTWVMALACAAPPLVGW |
| | PTH: | LWGFTVFGWGLPAVFVAVWVSVR |
| TM5 | OPS | SFVJYMFVVHFITPLIVIFFCYGQLVFTV |
| | PTH: | GNKKIIWHQVPILASIVLNHLFINIVRVL |
| TM6 | OPS | VTRMVIIMVIAFLICWLPYAGVAFYIIFT |
| | PTH: | LLKSTLVLMiPLFGVHYTVFMATPYTEVS |
| TM7 | OPS | IFMTIPAFFAKTSAVYNPVTYIMMNK |
| | PTH: | VQMHYEMLFNSFQGFFVAIIYCFCNG |

It was now possible to align receptors from Class A and B so as to maximize the evolutionary rank order correlation with GPCRs from Class A. The result was shown in Table III, which described the proposed aligmnent between bovine visual rhodopsin and the human parathyroid hormone receptor (PTH). As shown in FIG. 7B, these alignments yielded a correlation between Class B and Class A receptors over all seven helices that was comparable to that among Class A receptors. More over, alternative alignments shifted by up to ±4 residue positions yielded significantly smaller correlations.

Three lines of evidence supported this alignment. First, it maximized sequence identity between Class A and Class B GPCRs, despite their profound lack of sequence similarity. This was significant because rank order correlation and sequence identity were independent of each other, as established from the lack of correlation between the degree of identity of cognate residues and the extent to which their rank orders were correlated. Second, experiments based on the TM3 and TM6 alignments successfully reproduced double histidine mutations in the PTH receptor that had created a Zn-dependent switch in opsin. Third, the generic functional determinants of GPCR signaling predicted by a trace of Class A and B receptors, aligned as proposed, was consistent with the literature. Specifically, mutations at residues with rank-order below the 15^{th} percentile disrupt signaling in a multitude of GPCRs, whereas mutations of residues ranked above the 85^{th} percentile had a few consequences and these were ligand specific.

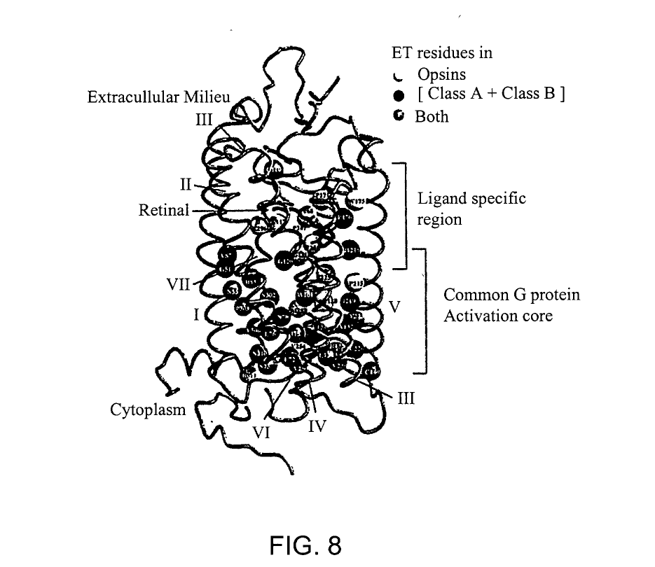

Lastly, it was possible to compare generic versus specific determinants of signaling. This was shown in FIG. 8, where the 15% best-ranked residues from opsins, adrenergic, chemokine, olfactory, and secretin-related receptors taken together and from visual rhodopsin alone are mapped into the Cα trace of rhodopsin (Palczewski et al., 2000) in black and white, respectively, with overlaps in gray. Most trace residues were internal, and concentrated toward the cytoplasmic, G-protein-coupling side of the transmembrane domain. Remarkably, class-specific residues that were unique to visual rhodopsins (white) clustered around the retinal and then formed a pathway extending toward the G protein through a series of van der Waals interactions between residues from TM3 and TM6. At the cytoplasmic third of the TM domain, this pathway spread out into an intricate network of interactions involving residues from TM1 (N55), TM2, (N73, 175, L76), TM3 (E134, R135), TM6 (V254, P267), and TM7 (N302, P303, Y306, N310). Nearly all these residues were functionally important globally as well, and as expected, this gray cluster was immediately adjacent to the extracellular loops of rhodopsin, consistent with an intimate role in G protein activation.

### Example 7

### Protein Test Set

The 46 proteins in the test set were listed in Table IV. In addition to the full name of the protein, also listed for each were the PDB identifier, class, size (amino acids), known function, number of sequences in the multiple sequence alignment, minimum percent identity between sequences in the alignment and the selected protein structure, the best significance level the protein achieves using the number of clusters statistical method, the best significance level the protein achieves using the size of largest cluster' method when gaps were considered as informative, and the evolutionary breadth of the protein family tree (E=eukaryotic, P=prokaryotic, V=viral).

Table IV
Summary of the 46 proteins in the test set.

| | | | | | | | | Best significance | | | | | |
|---|---|---|---|---|---|---|---|---|---|---|---|---|---|
| | | | | | | | | No. of clusters | | | Best significance: | | |
| | | | | | | No. of | | | | | Cluster Size | | |
| | PDB | | Evolutionary | | Protein | seq. in | Min % | Significance | Percent | Numerical | Significance | Percent | Numerical |
| Name | code | i) Function | Breath | SCOP Class | Size | alignment | identity | level (%) | coverage | value | level (%) | coverage | value |
| Ligand binding domain of | 1aij | LDL receptor | E | small | 37 | 184 | 46 | 30 | 25 | 2 | 0.3 | 30 | 8 |
| LDL receptor | | | | proteins | | | | | | | | | |
| c-Src tyrosine kinase; SH3 | 1nlo | Tyrosine Knase | E | β | 56 | 71 | 37 | 5 | 20 | 1 | 0.3 | 30 | 11 |
| Biotinyl domain | 1bdo | Carboxylase | E + P | β | 80 | 37 | 45 | >30 | | | 20 | 15 | 8 |
| Acyl CoA binding protein | 1aca | Binding protein | E | α | 86 | 38 | 46 | 5 | 20 | 3 | 0.3 | 30 | 24 |
| c-Src tyrosine kinase; SH2 | 1a09 | Tyrosine Kinase | E | α & β | 106 | 137 | 34 | 1 | 20 | 2 | 0.3 | 30 | 30 |
| Bikunin | 1 bik | Kunitz type inhibitor | E | Small | 110 | 36 | 43 | 5 | 20 | 4 | 10 | 20 | 15 |
| | | | | proteins | | | | | | | | | |
| Mannose binding protein | 2msb | Binds Mannose | E | α & β | 113 | 71 | 34 | 5 | 10 | 3 | 0.3 | 30 | 31 |
| Trp1 domain of Hop | 1elw | Chaperone | E | α | 117 | 42 | 41 | 1 | 10 | 3 | 1 | 10 | 9 |
| Pseudoazunin | 1bqk | Electron transport | E + P | β | 124 | 29 | 37 | 1 | 15 | 4 | 10 | 20 | 15 |
| Tpr2a domain of Hop | 1elr | 6Chaperone | E + P | α | 128 | 41 | 30 | 0.3 | 25 | 2 | 1 | 5 | 5 |
| Regulator of G-protein | 1fqi | Regulator of G-protein | E | α | 133 | 43 | 43 | 0.3 | 25 | 1 | 0.3 | 25 | 33 |
| signaling | | signaling | | | | | | | | | | | |
| Galectin-3 CRD | 1a3k | Galectin carbohydrate | E | β | 137 | 70 | 32 | 1 | 10 | 4 | 1 | 10 | 9 |
| | | recognition domain | | | | | | | | | | | |
| Myoglobin | 1a6m | Oxygen transport | E | α | 151 | 171 | 35 | 5 | 30 | 3 | 0.3 | 30 | 42 |
| Thermosome | 1ass | Chaperonin | E + P | α & β | 152 | 84 | 36 | 5 | 25 | 5 | 10 | 20 | 17 |
| Poly-A binding protein | 1cvj | Gene regulation | E α & β | 169 | 73 | 26 | 0.3 | 25 | 3 | 0.3 | 25 | 38 | |
| Growth hormone | 1a22-A | Growth hormone | E | α | 180 | 67 | 36 | 0.3 | 20 | 4 | 0.3 | 30 | 51 |
| Growth hormone receptor | 1a22-B | Growth hormone | E | α | 192 | 21 | 30 | 1 | 20 | 6 | 0.3 | 20 | 28 |
| | | receptor | | | | | | | | | | | |
| Astacin | 1ast | Metalloproteinase | E | α & β | 200 | 38 | 44 | 0.3 | 25 | 3 | 0.3 | 25 | 47 |
| | | (hydrolase) | | | | | | | | | | | |
| von Willebrand factor | 1 auq | blood coagulation | E | α & β | 208 | 44 | 34 | 5 | 5 | 5 | 1 | 5 | 6 |
| HSP-90 | 1am1 | Chaperone | E + P | α & β | 213 | 78 | 55 | 0.3 | 30 | 3 | 0.3 | 30 | 61 |
| Glutathione S-transferase, | 1aw9 | Transferase | E + P | α | 216 | 86 | 30 | 5 | 10 | 9 | 5 | 10 | 8 |
| type-III | | | | | | | | | | | | | |
| Adenylate kinase | 1aky | Phosphotransferase | E + P | α & β | 218 | 42 | 45 | 0.3 | 25 | 5 | 0.3 | 25 | 47 |
| F-MuLV | 1aol | viral glycoprotein | V | β | 228 | 21 | 39 | 0.3 | 25 | 5 | 0.3 | 25 | 54 |
| Estrogen receptor | 3ert | Nuclear receptor | E | α | 247 | 93 | 44 | 0.3 | 25 | 2 | 0.3 | 25 | 60 |
| Indole-3-glyceophosphate | 1a53 | Synthase | E + P | α & β | 247 | 19 | 30 | 0.3 | 20 | 3 | 0.3 | 30 | 70 |
| synthase | | | | | | | | | | | | | |
| Triosephosphate | 1amk | Gluconeogenesis | E + P | α & β250 | 73 | 47 | 0.3 | 25 | 6 | 0.3 | 25 | 52 | |
| isomerase | | | | | | | | | | | | | |
| Cyclins | 1fin-B | Transferase E | α | 260 | 23 | 34 | 0.3 | 30 | 4 | 0.3 | 30 | 69 | |
| Beta-Lactamase | 1btl | Hydrolase | P | Multi- | 263 | 50 | 45 | 0.3 | 20 | 9 | 0.3 | 25 | 54 |
| | | | | domain | | | | | | | | | |
| | | | | proteins | | | | | | | | | |
| Deacetoxycephalosporin C | 1rxg | Oxidoreductase | E + P | β | 275 | 24 | 25 | 0.3 | 15 | 8 | 1 | 15 | 19 |
| 2,5-diketo-D-gluconic acid | 1a80 | Oxidoreductase | E + P | α & β | 277 | 83 | 46 | 0.3 | 30 | 4 | 0.3 | 30 | 77 |
| reductase A | | | | | | | | | | | | | |
| Endonuclease IV | 1qum | Endonuclease | E + P | α & β | 279 | 27 | 39 | 0.3 | 20 | 5 | 0.3 | 20 | 53 |
| Dihydropteroate synthase | 1aj2 | Synthase | P | α & β | 282 | 42 | 37 | 0.3 | 20 | 6 | 0.3 | 25 | 60 |
| Protein phospatase-1 | 1fjm | Hydrolase | E | α & β | 294 | 68 | 65 | 0.3 | 30 | 3 | 0.3 | 30 | 87 |
| Signal sequence | 1ng1 | | E + P | α | 294 | 73 | 45 | 0.3 | 15 | 6 | 0.3 | 30 | 79 |
| recognition protein | | | | | | | | | | | | | |
| Cyclins | 1fin-A | Transferase E | α | 298 | 37 | 64 | 0.3 | 30 | 4 | 0.3 | 30 | 84 | |
| Thioredoxin reductase | 1f6m | Reductase | E + P | α & β | 320 | 44 | 56 | 0.3 | 25 | 8 | 0.3 | 30 | 86 |
| Annexin III | 1axn | calcium/phospholipid | E | α | 323 | 70 | 40 | 0.3 | 20 | 11 | 5 | 20 | 31 |
| | | binding protein | | | | | | | | | | | |
| Transferrin | 1a8e | iron transport | E | α & β | 329 | 52 | 46 | 0.3 | 20 | 6 | 0.3 | 15 | 26 |
| Peroxidase | 1aur | Peroxidase | E | α | 336 | 29 | 55 | 0.3 | 15 | 13 | 0.3 | 30 | 90 |
| Rhodopsin | 1f88 | Signaling protein | E | Membrane | 338 | 59 | 33 | 0.3 | 30 | 4 | 0.3 | 30 | 96 |
| | | | | and cell | | | | | | | | | |
| | | | | surface | | | | | | | | | |
| | | | | protein | | | | | | | | | |
| Serine/Threonine | 1a6q | Hydrolase | E | α & β | 363 | 58 | 38 | 0.3 | 20 | 8 | 0.3 | 15 | 31 |
| phosphatase | | | | | | | | | | | | | |
| citrate synthase | 1a59 | Synthase | E + P | α | 377 | 63 | 32 | 0.3 | 20 | 14 | 0.3 | 25 | 74 |
| Phosphoglycerate kinase | 16pk | Kinase | E + P | α & β | 415 | 95 | 41 | 0.3 | 25 | 11 | 0.3 | 25 | 93 |
| Alpha amylase | 1bag | alpha-amylase | E + P | β | 425 | 55 | 24 | 0.3 | 30 | 8 | 0.3 | 30 | 116 |
| HIV Reverse transcriptase | 1c1b | reverse transcriptase | V | α & β | 536 | 278 | 61 | 1 | 20 | 23 | 5 | 20 | 43 |
| Pyruvate decarboxylase | 1pvd | Carbon-Carbon lyase | E + P | α & β | 537 | 43 | 37 | 0.3 | 25 | 11 | 0.3 | 25 | 114 |

### Example 8

### QET Clustering Analysis

The quantitative significance of ET analysis was obtained by comparing clusters formed by trace residues to clusters created when an equal number of residues were randomly chosen.

Briefly, 12 proteins [1bdo (80 residues), 1a09 (106 residues), 1elw (117 residues), 1a6m (151 residues), 1ass (152 residues), 1am1 (213 residues), 1ao1 (228 residues), 1amk (250 residues), pp1 (294 residues), 1axn (323 residues), 1bag (425 residues), 1pvd (537 residues); full names are given in Table IV] were included in the test set for random cluster simulations based on the criteria that their sizes should adequately sample the region between a small protein (˜-80 residues) and a large protein (˜500 residues) and that their shapes are mostly globular. For each protein, the fraction of residues chosen randomly was determined as a percentage of the total number of residues present in that protein, beginning with 5% and increasing in increments of 5% to 95% (although only coverages up to 30% shown herein). At each coverage level, individual residues were selected randomly and both the total number of clusters and the size of each cluster were recorded. This process was repeated 5000 times (a compromise between statistical significance and computational time) for each protein at each coverage level to generate the complete data set for further analysis.

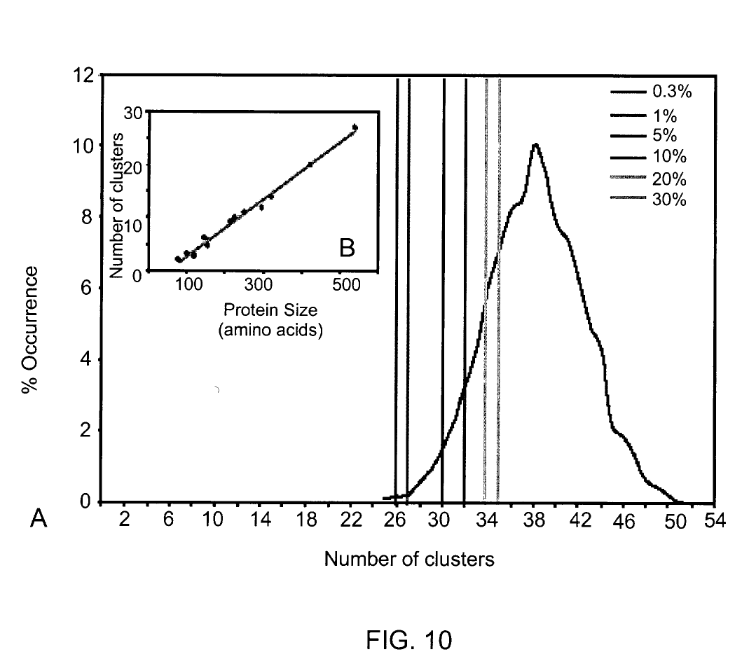

The randomly selected residues were defined as a cluster if any atom in one residue was within 4 Å of any other atom in another residue (hydrogen atoms excluded). The typical distribution of the number of clusters followed a long-tailed distribution as shown in FIG. 10. The number of clusters were calculated at threshold values of 0.3%, 1%, 5%, 10%, 20% and 30% significance. 0.3% significance, for example, implied that the probability of randomly observing the corresponding number of clusters was 3 in 1000.

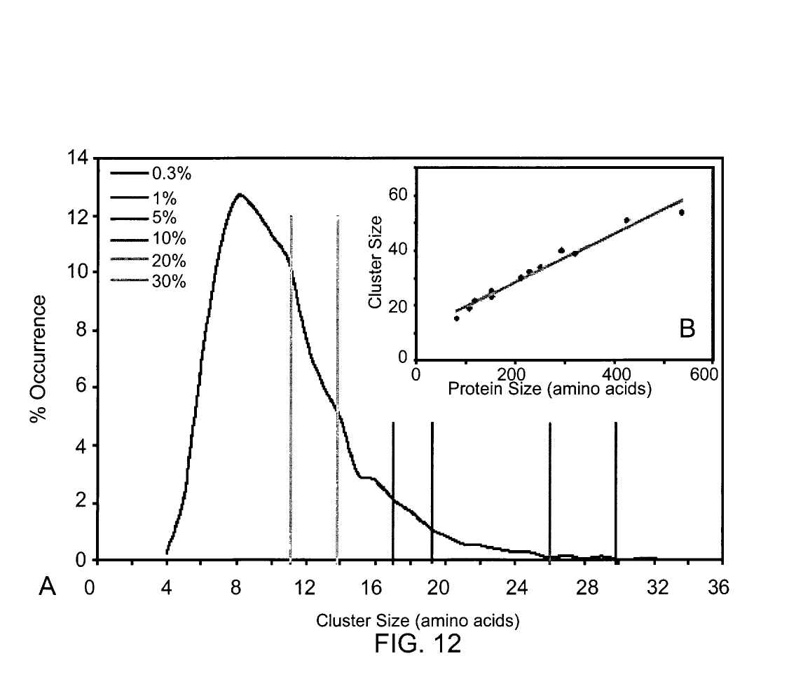

For each of the 5000 iterations, at each coverage level, the size of the largest cluster was recorded and their distribution was plotted for each coverage level (FIG. 12). The resulting distributions closely resembled those observed for the number of clusters analysis and therefore a similar approach of determining threshold values was used. However, the tail of the distribution corresponding to a larger value (of cluster size) was used rather than a smaller one as before, although for the same reason (signal is defined as a small number of large clusters).

Random distributions were established in 12 proteins ranging from 80 to 537 residues in length. For each, a number of residues representing 5%, 10%, 15%, 20%, 25%, and 30% coverage were picked randomly and repeatedly 5000 times. Using random distribution, it was determined whether an observed number of clusters was consistent with the null hypothesis that they arose by chance (H₀), and, if not, determine the specific level of confidence at which the null hypothesis is rejected.

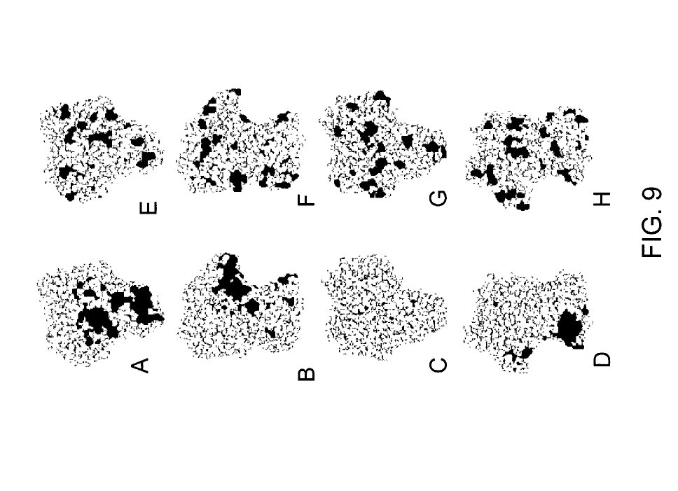

A trace of pyruvate decarboxylase (1pvd) revealed two large clusters confined predominantly to one face of the protein, as shown in FIG. 9, where FIG. 9A to FIG. 9D were rotated by increments of 90° about a vertical axis. In contrast, a random sampling of the same number of residues yielded many smaller clusters distributed uniformly over the surface, as shown in FIG. 9E-FIG. 9H.

As shown in pyruvate decarboxylase (1pvd), FIG. 10, when 81 of its 537 residues were picked randomly over 5000 trials (for a coverage of nearly 15%), the number of clusters formed each time forms a histogram distributed unevenly about 38, with extremes ranging from 25 to 55. In 99.7% of the trials, the number of generated clusters was greater than 26, so that if a trace yielded fewer than 26 clusters, the Ho is rejected at a significance level (p-value) of 0.3%. Similarly, the number of cluster thresholds to reach significance levels of 1%, 5% and 30% are 27, 30, and 35 respectively. The trace of pyruvate decarboxylase identified a total of 10 clusters and the probability that this reflects random chance is much less than 0.3%.

To further understand the significance of individual trace clusters, the size of the largest cluster, dominant cluster to the size expected to occur by chance, was determined. For this purpose, distributions of the largest cluster size use built using the method already described above, and shown in FIG. 12 for protein pyruvate decarboxylase (1pvd) at 15% coverage. Typically the largest cluster contained 8 residues, with sizes ranging from 4 to 34 residues over 5000 trials. In order to achieve a significance level of 30%, 5%, 1%, or 0.3%, the largest trace cluster would have to comprise at least 11, 19, 26, or 30 residues, respectively. In fact, the largest cluster traced in FIG. 12 included 74 residues, and thus achieved a significance much better than 0.3%.

Here again, for a given coverage and as shown in the FIG. 12B, the threshold for the size of the largest cluster needed to reach a given level of significance is nearly a linear function of protein size. This relationship held for all levels of significance up to 40%, and the high quality-of-fit R2 values (ranging from 0.77-0.97) allowed linear relationship to be applied to other proteins. For example, using the FIG. 12B showing the linear fit thresholds for 20% coverage at the 0.3% level of significance, a 250 amino acid protein would achieve significance at the 0.3% level if its largest cluster contained at least 33 residues.

### Example 10

### Linear Fitting

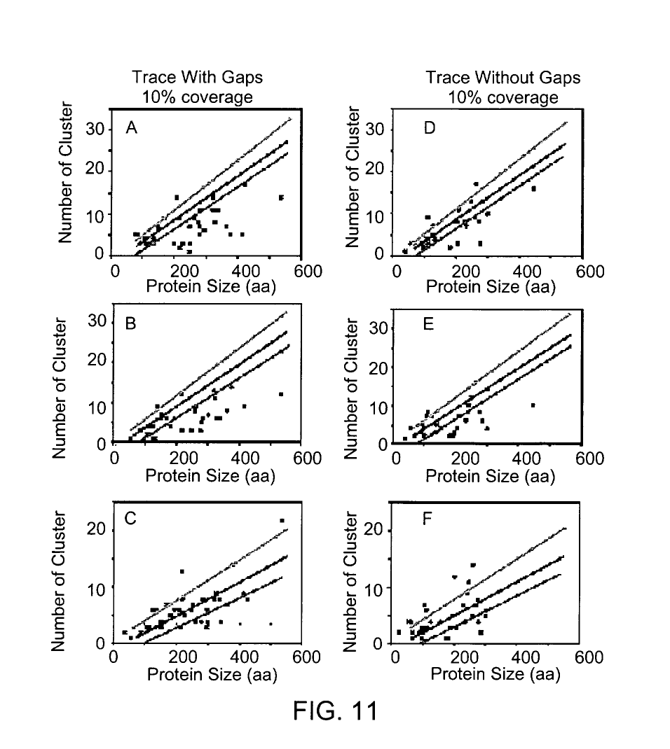

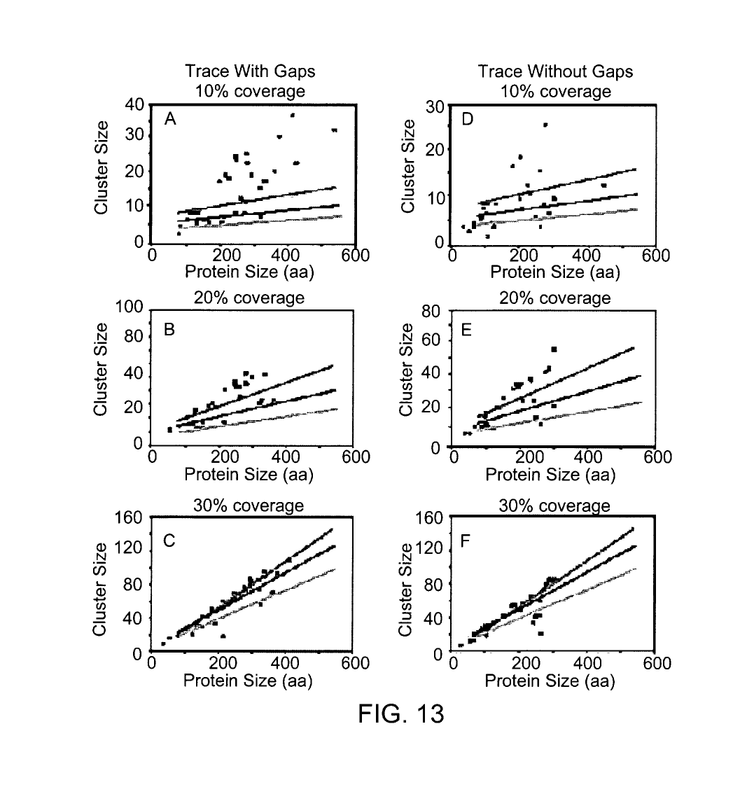

Both the threshold values for the number of clusters and the size of the largest cluster analyses followed a linear relationship with respect to protein size. In order to extrapolate the data to proteins of all sizes, each threshold value against protein size was plotted to allow comparison of an observed threshold value with the value expected randomly (and the significance of such a comparison) for a given coverage (FIGS. 11 and 13). Since a linear relationship was found irrespective of protein size, significance, or coverage, linear fits for each coverage and significance level were obtained and used to generate lookup tables whereby the fits at a given coverage and significance level is used to extrapolate a threshold value for a protein of any size within the range test. The statistics used to construct FIGS. 11 and 13 were discrete and not continuous as may be implied by the smooth envelope of the histogram. All the thresholds obtained by linear extrapolation were rounded off to the nearest integer.

For a given coverage, the threshold of the number of clusters needed to reach a given level of significance was nearly a linear function of protein. This was demonstrated in FIG. 10B, which showed the number of clusters corresponding to the 1% significance threshold for each of the 12 proteins as a function of their size, at 15% coverage. This relationship held for all of the defined significance levels at coverages ranging from 5% to 30% of the protein. In addition, the high quality-of-fit values, R2 (ranging from 0.89-0.99), suggest that the specific choice of these 12 proteins was unlikely to bias the results. Thus, instead of building individual clustering distributions for each protein under study, many computational cycles was saved by using a generic lookup table with the information from the linear fit in order to determine the significance level for an observed number of clusters identified by the trace. For example, using the FIG. 10B, if the top 55 trace residues in a 370 amino acid protein (˜15% coverage) formed 17 clusters or fewer, they would achieve a significance at the 1% level.

### Example 11

### Treatment of Gaps

Gaps were treated as if they were a twenty-first amino acid type. Here the convention that a gap was interpreted in the same way that Ala, Val, or any of the other 20 amino acids positions was not meant to carry biophysical meaning. It was simply a computational device, which was reasonable because gaps often occurred in blocks in a multiple sequence alignment. These blocks indicated that a deletion or insertion took place in a conserved region in all protein descendants, which suggested some functional importance at the location of those gaps. In practice, the ability to rank gapped positions eliminated “holes” from ET analyses allowing maximum coverage all the residues in the structure to be ranked.

To evaluate this new approach, the original, gap-intolerant method, where gapped positions were excluded from the trace and remain unranked was used and compared to the gap tolerant method.

### Example 12

### Quantitative Evolutionary Trace Analysis

A total of 46 proteins were selected from the PDB so that they represented a range of protein sizes (the smallest one 37 residues and the largest one 537 residues in length), a wide range of protein folds, and a diversity of biological function (Table IV). The multiple sequence alignments were generated using pileup (of GCG package) or CLUSTALW using their default variables and the trees obtained are rooted and unbalanced. Two different types of ET analyses were performed on each of these 46 proteins. The first method of analysis discounted any residue position containing a gap; while the second method of ET analysis included such residue positions by treating gaps in the sequence alignment as a twenty-first amino acid. By considering a gap as an artificial amino acid type, this allowed every residue in the structure to be assigned a rank (as opposed to the first method where gap positions are completely ignored), leading to 100% coverage of the protein structure at the maximum rank, and thus enabled direct comparison between ET predictions and the random clustering. The first method, however, required an adjustment to be made in the coverage values so as to account for the fact that at maximum rank, 100% coverage of the protein may not have been achieved. In both the cases input data set was not optimized (i.e. fragmented sequences, incomplete sequences, distant homologues, mutants, and sequences that vary in length from the sequence of the structure were not pruned from the input to the multiple sequence alignment). These sequences were not removed in order to mimic an input that could be fed into the program by a scientist not trained to use the QET.

### Example 13

### T Mapping

Ranks were converted into their corresponding coverage levels by dividing the number of class specific residues at a given rank by the total number of residues able to be assigned a rank. When residue positions containing gaps were excluded from trace analyses the total number of residues became the number of class specific residues found at the maximum rank. When gaps were treated as artificial amino acids, the total number of residues was simply the total number of residues in the protein structure. The significance of any rank was determined by examining where the observed number of clusters (at that rank's coverage) fell with respect to the significance thresholds established from the linear fitting of the random cluster data. In the case where gaps were excluded from the analyses, the significance of the ranks was determined in the same manner as when gaps were included, but the protein size was considered to be the maximum number of trace residues and not the total number of residues in the protein. Visualization of trace predictions were done using Rasmol molecule viewer.

### Example 14

### Statistical Significance

In the novel QET method in 46 proteins, it was found in all but 2 that the observed number of clusters reached a significance level of 5% or better for at least one level of coverage (96% success rate). In fact, almost two-thirds (30 out of 46, or 65%) of the traces reached a significance level of 0.3% or better, as shown in Table V. The first protein in which the trace failed to reach the 5% significance level was the ligand-binding domain of the LDL receptor. This trace involved essentially no pruning of its sequence family, so that 184 homologues were traced together, and the protein itself was very small (37 residues) which favored the natural clustering of randomly drawn residues. Interestingly, the cluster size statistic showed that this trace was nevertheless significant. The other trace that did not reach significance was the biotinyl domain involved in fatty acid synthesis. In comparison, the original gap-intolerant method yielded statistically significant traces in fewer cases. 33 out of 46 traces (or 72%, down from 96%) reached a significance level of 5% or better at least once (Table V) at some coverage between 5 and 30% (Table VI), and 26 achieved a significance level of 0.3% or better (or 57% down from 65%).

These results were further broken down for specific coverages. Thus, among the 22 proteins that had a rank corresponding to nearly 5% coverage, 45% were significant (the other 24 proteins had ranks that corresponded to coverages other than 5%). For the 33 proteins that had a rank near 10% coverage, 79% achieved were significance. At coverages of 15%, 20%, 25%, and 30%, the number of protein with matching ranks were 32, 32, 35, and 35, respectively, and the fractions that achieved the 5% significance level were 75%, 91%, 74% and 49%, respectively (Table VI).

Nearly similar results were obtained by considering the size of the dominant cluster. Using the novel trace approach, 42 of 46 proteins (91%) reached the 5% significance level, and 34 (74%) reached the 0.3% significance level, as shown in FIG. 13 and in Table V. As before, the statistics worsen somewhat when gaps were not counted in the trace. Nevertheless 36 (78%) of the 46 proteins reached the 5% significance level, and 29 (63%) were at the 0.3% significance level.

TABLE V

| | Number of Clusters | | | | Size of Largest Cluster | | | |
|---|---|---|---|---|---|---|---|---|
| | with gaps | | Without gaps | | With gaps | | Without gaps | |
| Significance | Number | | Number | | Number | | Number | |
| level | of | Fraction | of | Fraction | of | Fraction | of | Fraction |
| (%) | proteins | (%) | proteins | (%) | proteins | (%) | proteins | (%) |
| <0.3 | 30 | 65 | 26 | 57 | 34 | 74 | 29 | 63 |
| 0.3-1 | 6 | 13 | 3 | 7 | 5 | 11 | 6 | 13 |
| 1-5 | 8 | 17 | 4 | 9 | 3 | 7 | 1 | 2 |
| 5-10 | 0 | 0 | 6 | 13 | 3 | 7 | 2 | 4 |
| 10-20 | 0 | 0 | 2 | 4 | 1 | 2 | 1 | 2 |
| 20-30 | 1 | 2 | 1 | 2 | 0 | 0 | 4 | 9 |
| >30 | 1 | 2 | 3 | 7 | 0 | 0 | 2 | 4 |

As above, these results were further broken down in terms of the coverage as shown in Table VI. The number of proteins with matching ranks at 5, 10, 15, 20, 25 and 30% coverages were 22, 33, 32, 32, 35 and 35 respectively. Specifically, a significance level of 5% was achieved at 5% coverage in 50% of the proteins with matching ranks, 76% at 10% coverage, 72% at 15% coverage, 75% at 20% coverage, 57% at 25% coverage, and 71% at 30% coverage.

TABLE VI

| | Coverage | | | | | | | | | | | |
|---|---|---|---|---|---|---|---|---|---|---|---|---|
| Significance | 5% | | 10% | | 15% | | 20% | | 25% | | 30% | |
| level | No of | Fraction | No of | Fraction | No of | Fraction | No of | Fraction | No of | Fraction | No of | Fraction |
| (%) | proteins | (%) | proteins | (%) | proteins | (%) | proteins | (%) | proteins | (%) | proteins | (%) |
| <0.3 | 0 | 0 | 17 | 52 | 16 | 50 | 18 | 56 | 19 | 54 | 18 | 51 |
| 0.3-1 | 9 | 41 | 4 | 12 | 1 | 3 | 1 | 3 | 0 | 0 | 1 | 3 |
| 1-5 | 2 | 9 | 4 | 12 | 6 | 19 | 5 | 16 | 1 | 3 | 6 | 17 |
| 5-10 | 2 | 9 | 3 | 9 | 2 | 6 | 4 | 13 | 2 | 6 | 2 | 6 |
| 10-20 | 1 | 5 | 4 | 12 | 5 | 16 | 2 | 6 | 3 | 9 | 1 | 3 |
| 20-30 | 0 | 0 | 0 | 0 | 0 | 0 | 2 | 6 | 4 | 11 | 2 | 6 |
| >30 | 8 | 36 | 1 | 3 | 2 | 6 | 0 | 0 | 6 | 17 | 5 | 14 |
| Total | 22 | 100 | 33 | 100 | 32 | 100 | 32 | 100 | 35 | 100 | 35 | 100 |

### Example 15

### Signal to Noise Threshold

The ET signal-to-noise threshold varied among proteins. Initially, at top ranks, relatively few residues were class specific, therefore, coverage was low and the trace residues were too sparse to make direct contacts (within 4 Å), and thus they did not cluster significantly. Thus, as shown in Table VII, relatively few proteins achieved significance at 5% coverage. As the rank threshold was lowered, more residues became class specific and these tended to fill the gaps between top-ranked residues traced earlier, thereby coalescing many small clusters into fewer, larger ones. This reflected the tendency of ET clusters to expand outward from small cores of critically important residues. In keeping with this scenario, most traces reached significance between 15 to 25% coverage. Eventually, when coverage reaches 30% to 35%, so many residues were considered that they would cluster even when picked randomly, and the significance of trace clusters diminished as seen in FIG. 11 and in Table VII. The threshold at which the trace residue clusters ceased to be significant varied, most often in the 20% to 35% coverage range.

TABLE VII

| | Coverage | | | | | | | | | | | |
|---|---|---|---|---|---|---|---|---|---|---|---|---|
| Significance | 5% | | 10% | | 15% | | 20% | | 25% | | 30% | |
| level | No of | Fraction | No of | Fraction | No of | Fraction | No of | Fraction | No of | Fraction | No of | Fraction |
| (%) | proteins | (%) | proteins | (%) | proteins | (%) | proteins | (%) | proteins | (%) | proteins | (%) |
| <0.3 | 6 | 27 | 20 | 61 | 19 | 59 | 19 | 59 | 16 | 46 | 7 | 20 |
| 0.3-1 | 1 | 5 | 3 | 9 | 2 | 6 | 4 | 13 | 5 | 14 | 3 | 9 |
| 1-5 | 3 | 14 | 3 | 9 | 3 | 9 | 6 | 19 | 5 | 14 | 7 | 20 |
| 5-10 | 2 | 9 | 2 | 6 | 3 | 9 | 1 | 3 | 2 | 6 | 10 | 29 |
| 10-20 | 1 | 5 | 1 | 3 | 2 | 6 | 0 | 0 | 2 | 6 | 0 | 0 |
| 20-30 | 3 | 14 | 0 | 0 | 1 | 3 | 1 | 3 | 3 | 9 | 2 | 6 |
| >30 | 6 | 27 | 4 | 12 | 2 | 6 | 1 | 3 | 2 | 6 | 6 | 17 |
| Total | 22 | 100 | 33 | 100 | 32 | 100 | 32 | 100 | 35 | 100 | 35 | 100 |

### Example 16

### Ligand Contacts

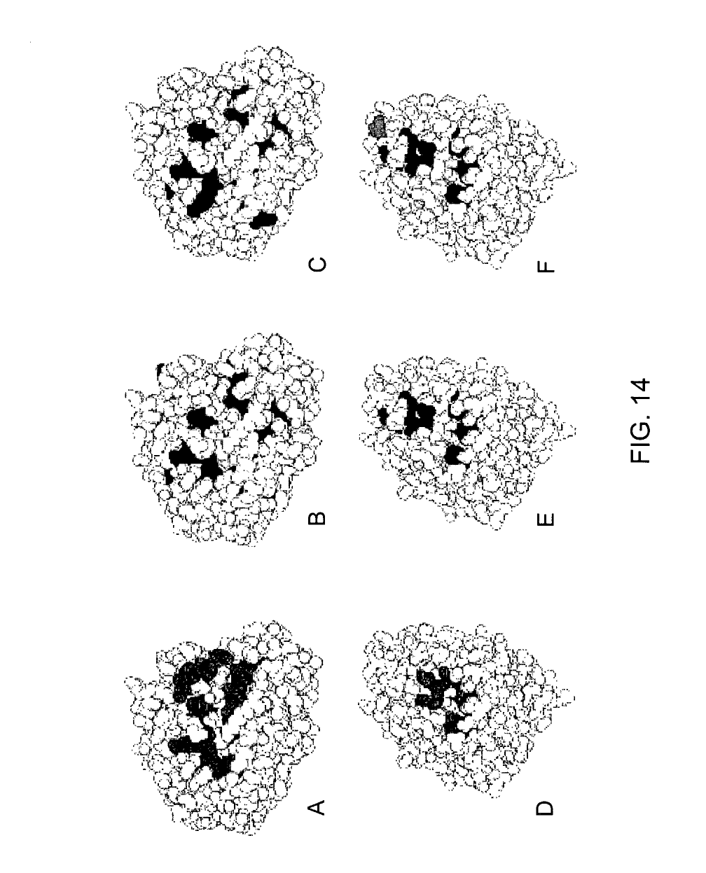

Among the 38 structures in the test set that had bound ligands, 36 (95%) traces reached significance with the new method, compared to 30 (79%) which reached significance with the gap intolerant approach. In one more protein, HIV reverse transcriptase ET identified a few residues contacting the ligand, but the clusters were not significant. However, with additional pruning of the original 278 sequences, that trace became significant as well. In all but one of these cases, the known ligand(s) directly contacted a trace cluster as shown for 2 representative traces in FIG. 14. ET identified some but not necessarily all of the residues contacting the ligand, consistent with the fact that not all interfacial residues were important for ligand binding. This was also a function of the rank, or coverage since top ranked residues represent the most critical determinants of molecular recognition, while somewhat lesser ranked residues may play a role in specificity. The overall results for the 38 proteins having bound ligands are shown in Table IV. Despite the lack of optimization of these traces, it is noteworthy that ET provides biologically relevant information in 37 out of 38 cases, and in 36 of those cases, that information is also statistically significant.

### Example 17

### Cluster-Based Overlap Statistics

The overlap of trace clusters and functional sites were examined in 86 proteins drawn from three data sets. For lack of direct experimental evidence, the functional site was defined in many cases as all residues with a non-hydrogen atom within 5 angstroms of a ligand. Thus, the “protein-ligand” set consisted of the 37 protein-ligand complexes out of a larger set of 46 previously defined in Example 7. Such structurally determined functional sites were large, with an average of 28±7 residues representing 13±3% of the protein. By contrast, in the “enzyme” set of representatives from 29 superfamilies defined by Todd et al. (2001) individual active site residues were known experimentally from the literature. These sites were small with only 4 residues on average, or about 1% of the protein. As a further objective test of QET, the “SGI” set consisted of the 22 protein-ligand complexes out of the 42 structures solved in the context of the SGI and readily accessible in the PDB (Berman et al., 2000). Again, for lack of direct biochemical evidence, the functional sites were defined by proximity to a ligand. This set was further reduced to 20 proteins after removal of two outliers: the subunits in the Cyanate Lyase complexes (PDB codes 1dwk and 1dw9) whose decameric arrangement yields a functional site covering 72% of the protein, far more than the average 10±3% (21±8 residues).

Quantitative Evolutionary Trace was performed as described in Examples 12, 13 and 14. In this study, trace clusters were considered significant if both the number of clusters and size of largest cluster occurred in less than 5% of the random simulations.

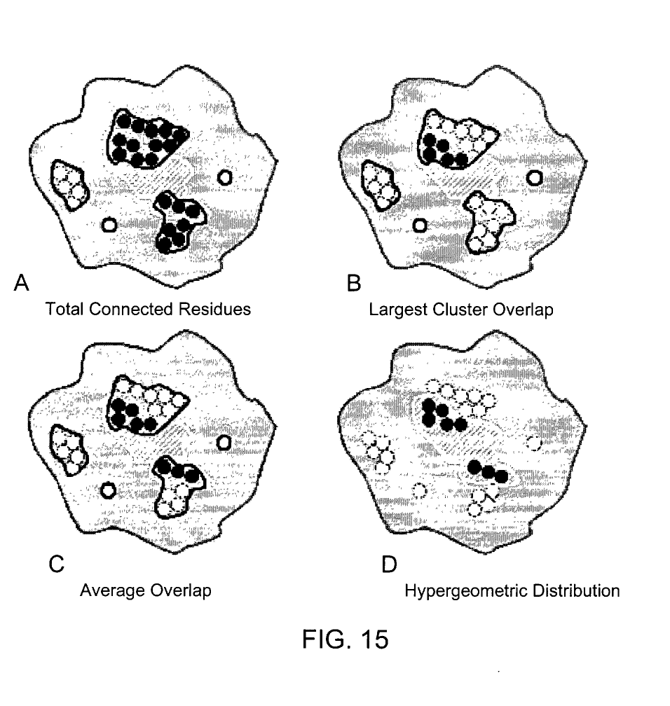

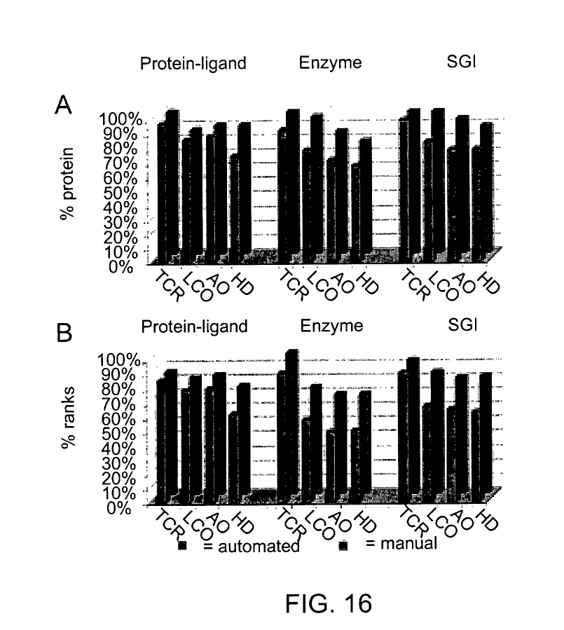

In order to determine whether trace residues that form significant structural clusters identify functional sites accurately, these proteins were used to measure the statistical significance of the overlap between such trace clusters and functional sites defined by ligand proximity (protein-ligand and SGI sets) or experiment (enzyme set). The first statistic, Total Connected Residues (FIG. 15A), counted as a success any trace residue in any cluster that overlaps with the functional site. This may be overly generous but it was consistent with functional sites that extended beyond a ligand's immediate vicinity (Lichtarge et al., 2002; Lockless et al., 1999). By this measure, there was always at least one rank at which the trace clusters overlapped significantly with the functional sites for all 79 proteins (FIG. 16A, black bars).

The second statistic, the Largest Cluster Overlap statistic (FIG. 15B), counted as a success only those residues that were both in the largest cluster and in the functional site. Despite the stringency of this definition, the overlap was significant for at least one rank in 88%, 97%, and 100% of the protein-ligand, enzyme, and SGI data sets, respectively.

The third statistic, the Average Overlap statistic (FIG. 15C), counted as a success all trace residues that overlap with the functional site, but it penalized predictions with multiple overlaps due to distinct clusters. Again significance was high: 91%, 86%, and 94% in the same sets.

Finally, the Hypergeometric Distribution statistic (FIG. 15D) counted as a success only trace residues directly part of the functional site. By this measure, other top-ranked residues that extended this site or was important for structural and dynamical properties counted as failures, regardless of the cluster they belong to. For this reason, this statistic was likely to systematically underestimate the true significance of overlap and to provide a lower bound for the cluster-based ones. Even so, it indicated significant overlaps for 91%, 79%, and 89% of the protein-ligand, enzyme, and SGI sets, respectively.

### Example 18

### Automated QET Versus Manuel QET

ET's performance was tested without manual pruning of sequence fragments and of evolutionary outliers from the input. This automated trace identified statistically significant structural clusters of evolutionarily important residues in 81/86 (94%) of the proteins (35/37, 28/29, and 18/20 in the protein-ligand, enzyme, and SGI datasets, respectively as described in Example 17). Of these 81 proteins, the fraction that achieved significant overlap for at least one rank was 97%, 93%, and 100% for the protein-ligand, enzyme, and SGI sets, as measured by the Total Connected Residues statistic (FIG. 16A, gray bars). Thus, the automated trace correctly identified functional sites in 78 of the 81 proteins (96%) by the most favorable statistic. This success rate was 83% by the Largest Cluster Overlap, 80% by the Average Overlap, and 73% using the lower-bound estimate of the Hypergeometric Distribution. The percentage of significant ranks that had significant overlap, averaged by dataset, was: protein-ligand=85%, enzyme=90%, SGI=90%, using Total Connected Residues; FIG. 16B, gray bars). Thus, among the 86 proteins tested, the automated ET identified functional sites in 69% according to the least favorable statistic and in 91% according to the most favorable one, suggesting that ET can be made applicable to the proteome at large.

### Example 19

### Accuracy to Functional Site Identification

A complementary approach to measure the accuracy of functional site identification was to determine how much of the functional site is identified by the largest trace cluster when the trace reached its signal to noise rank threshold.

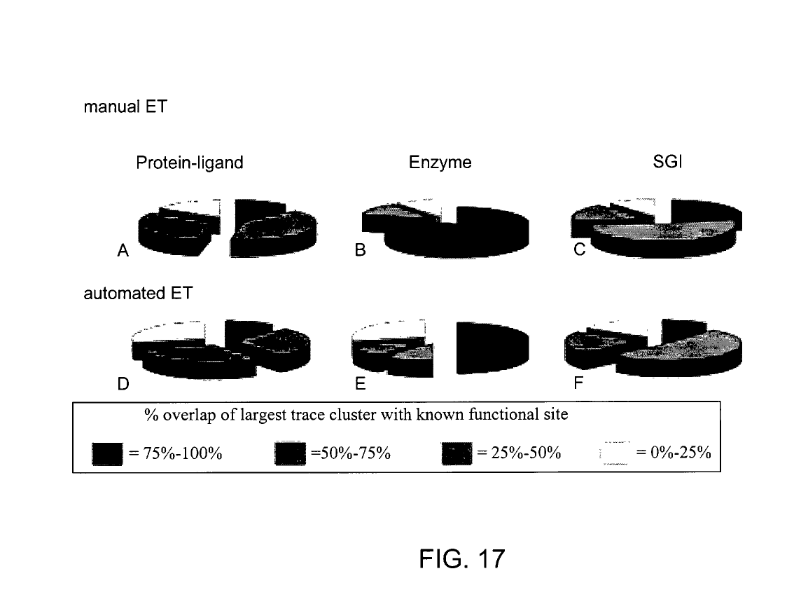

For manually optimized traces and among the 79 proteins with significant clusters which are described above in Examples 17 and 18, more than 50% of the site was traced in 53% of the protein-ligand set, in 90% of the enzyme set, and in 72% of the SGI set (FIGS. 17A-17C). These variations arose from basic differences between functional sites defined by ligand proximity (ligand and SGI sets) and those defined experimentally (enzyme set). In the latter, active sites were mostly limited to key catalytic residues. This was only 4 residues on average but it was as few as 1, such as in chloramphenicol acetyltransferase (PDB code 1xat, EC number 2.3.1.28) where His 79 is the lone putative general base for hydroxyl deprotonation (Todd et al., 2001). Trace clusters overlapped most of these residues, but because they were so few, the overlap tended to be less statistically significant. On the other hand, ligand proximity was only a rough measure of the true functional site since only a fraction of the residues near a ligand contribute to binding (Cunningham et al., 1993). In that case, the overlap in the protein-ligand and SGI data sets tended to be less extensive but more significant. At worst, the largest cluster covered less than 25% of the functional site in 16% of the protein-ligand set, 10% of the enzyme set, and in 11% of the SGI set (FIGS. 17A-17C). When the automated ET was used, the percentage of the 81 proteins with significant clusters that had greater than 50% overlap with the functional site was 40% in the protein-ligand set, 61% in the enzyme set, and 61% in the SGI set (FIGS. 17D-17F). Thus, automated ET accurately identified significant trace clusters in 35 of the 40 SGI proteins.

### References Cited

All patents and publications mentioned in the specification are indicative of the level of those skilled in the art to which the invention pertains. All patents and publications are herein incorporated by reference to the same extent as if each individual publication was specifically and individually indicated to be incorporated by reference.

Aksentijevich et al., Human Gene Ther. 7:1111, 1996.

Angleson, J. and T. G. Wensel, J. Biol. Chem. 269, 16290 (1994).

Arshavsky, V. Y. and M. D. Bownds, Nature 357, 416 (1992).

Baranski, T. J., et al., (1999). J. Biol. Chem. 274, 15757-15765.

Benzing, T. et al., J. Biol. Chem. 275 28167 (2000).

Berman, D. M. and A. G. Gilman, J. Biol. Chem. 273, 1269 (1998).

Berman, D. M. et al., T. Kozasa, and A. G. Gilman, J. Biol. Chem. 271, 27209 (1996).

Casari, G. et al., (1995). Nature Structural Biology 2, 171-178.

Cunningham, B. C. & Wells, J. A. (1993). J. Mol. Biol. 234, 554-63.

Druey, K. M. et al., Nature 379, 742 (1996).

Feigner et al. Proc. Nat'l Acad. Sci. USA 84:7413, 1987.

Felsenstein, J. Cladistics 5, 164 (1993).

Feng et al., J. Mol. Biol. 25:351-360, 1987.

Fraley et al., Proc. Nat'l Acad. Sci. USA, 76:3348-3352, 1979.

He, W. et al., Neuron 20, 95 (1998).

Hellinga, H. W. and J. S. Marvin, Trends Biotechnol. 16, 183 (1998).

Henikoff, S. and Henikoff, J. G. (1991). Nucleic Acids Res 19(23), 6565-6572.

Higgins et al., Comput. Appl. BioSci. 5:151-153, 1989.

Johannesson et al., 1999, J. Med. Chem. 42:601-608.

Johnson et al., Peptide Turn Mimetics” IN: Biotechnology And Pharmacy, 1993.

Jones, S. and J. M. Thornton, J. Mol. Biol. 272, 121 (1997).

Kostenis, E. et al., Biochemistry 36, 1487 (1998).

Kovoor, A. et al., Biol. Chem. 275, 3397 (2000).

Kuntz, I. D. et al., (1982). J Mol Biol 161, 269-288.

Lamb, M. L., and Jorgensen, W. L.. (1997). Curr Opin Chem Biol. 1, 449-457.

Lambright, D. G. et al., Nature 379, 311 (1996).

Lichtarge, O. et al., J. Mol. Biol. 257, 342 (1996).

Lichtarge, O. et al., (1996). Proc. Nat'l Acad. Sci. USA. 93, 7507-7511.

Lichtarge, O. et al., (1997). J Mol Biol 274, 325-337.

Lockless, S. W. & Ranganathan, R. (1999). Science. 286, 295-299.

Ma, B. et al., (2001). Curr Opin Struct Biol 11(3), 364-9.

McElroy, H. E., et al., J. Cryst. Growth. 122:265-272, 1992.

Miranker, A. et al., (1991). Proteins 11, 29-34.

Natochin, M. et al., J. Biol. Chem. 273, 6731 (1988).

Noel, J. P. et al., Nature 366, 654 (1993).

Onrust, R. et al., (1997). Science 275(5298), 381-4.

Pearce, K. H. et al., Biochemistry 35, 10300 (1996).

Shatsky, M. F. et al., in Intell Syst Mol Biol Vol. 8 329-343 (2000).

Slep, K. C. et al., (2001). Nature 409(6823), 1071-7.

Sowa, M. et al., Nat. Struct. Bio. 8, 234 (2001).

Sowa, M. E. et al., (2000). Proc. Nat'l. Acad. Sci. USA 97(4), 1483-8.

Taylor, J. M. et al., J. Biol. Chem. 269, 27618 (1994).

Tesmer, J. J., et al., Cell 89, 251 (1997).

Thompson, J. et al., Nucleic Acids Res. 22, 4673 (1994).

Todd, A. E. et al., J. Mol. Biol. 307,1113-1143.

Watson, N. et al., Nature 383, 172 (1996).

Weisshoff et al., 1999, Eur. J. Biochem. 259:776-788.

Wu, G. et al., J. Biol. Chem. 273,7197 (1998).

Yao et al., in press

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

We claim:

1. A method of determining a functional site in a protein comprising the steps of:
obtaining a protein sequence;
aligning the protein sequence to homologous protein sequences to generate a multiple sequence alignment;
adding gap tolerance, wherein a gap in the protein sequence alignment is considered as an artificial amino acid;
producing an evolutionary trace, wherein the evolutionary trace identifies residues that are trace residues; and
determining cluster formation of the trace residues, wherein a cluster indicates the functional site of the protein.

2. The method of claim 1, wherein the determining step comprises mapping the class-specific residues onto a representative protein structure and examining for cluster formation.

3. The method of claim 1, wherein the obtaining step comprises obtaining the protein sequence and homologous protein sequences.

4. The method of claim 1 further comprising determining clustering statistics.

5. The method of claim 4, wherein the clustering statistics comprises the overall number of clusters.

6. The method of claim 4, wherein the clustering statistics comprises the size of the largest cluster.

7. The method of claim 4, wherein the clustering statistics comprises the overall number of clusters and the size of the largest cluster.

8. A method of determining a functional site in a protein comprising the steps of:
obtaining a protein sequence;
aligning the protein sequence to homologous protein sequences to generate a multiple sequence alignment;
adding gap tolerance, wherein a gap in the protein sequence alignment is considered as an artificial amino acid;
producing an evolutionary trace, wherein the trace identifies residues that are trace residues;
determining cluster formation of the residues;
performing clustering statistics, wherein the statistics indicates the functional site of the protein; and
mapping the trace residues.

9. The method of claim 8, wherein the clustering statistics comprises the overall number of clusters.

10. The method of claim 8, wherein the clustering statistics comprises the size of the largest cluster.

11. The method of claim 8, wherein the clustering statistics comprises the overall number of clusters and the size of the largest cluster.

12. A protein database comprising proteins having predicted functional sites.

13. The database of claim 12, wherein the database is produced using evolutionary trace analysis having gap tolerance.

14. The database of claim 12, wherein the database is produced using evolutionary trace analysis and clustering statistics.

15. The database of claim 12, wherein the database is produced using evolutionary trace analysis having gap tolerance and clustering statistics.

16. A method of producing a protein database having predicted functional sites comprising the steps of:
obtaining a protein sequence;
aligning the protein sequence to homologous protein sequences to generate a multiple sequence alignment;
adding gap tolerance, wherein a gap in the protein sequence alignment is considered as an artificial amino acid;
producing an evolutionary trace, wherein the trace identifies residues that are trace residues; and
determining cluster formation of the residues, wherein a cluster indicates the functional site of the protein.

17. The method of claim 16 further comprising determining clustering statistics.

18. The method of claim 17, wherein the clustering statistics comprises the overall number of clusters.

19. The method of claim 17, wherein the clustering statistics comprises the size of the largest cluster.

20. The method of claim 17, wherein the clustering statistics comprises the overall number of clusters and the size of the largest cluster.

21. A peptide database comprising peptides which are the binding sites of proteins.

22. The database of claim 21, wherein the database is produced using evolutionary trace analysis.

23. The database of claim 21, wherein the database is produced using evolutionary trace analysis having gap tolerance.

24. The database of claim 21, wherein the database is produced using evolutionary trace analysis having gap tolerance and clustering statistics.

25. The database of claim 21, wherein the database is produced using evolutionary trace analysis and clustering statistics.

26. The database of claim 21, wherein the binding sites are the ligand binding pockets of G-protein coupled receptors.

27. A method of producing a peptide database having peptides that are the binding sites of proteins comprising the steps of:
obtaining a peptide sequence;
aligning the peptide sequence to homologous peptide sequences to generate a multiple sequence alignment;
adding gap tolerance, wherein a gap in a peptide sequence alignment is considered as an artificial amino acid;
producing an evolutionary trace, wherein the trace identifies residues that are trace residues; and
determining cluster formation of the residues, wherein a cluster indicates the binding site.

28. A method of aligning remote protein homologs comprising the steps of:
obtaining protein sequences of at least two proteins with no sequence homology;
producing a separate evolutionary trace sequence of each protein, wherein the evolutionary trace sequence identifies residues that are trace residues;
assigning evolutionary rank to trace residues from each trace;
assigning an order based on the evolutionary rank;
determining a correlation between any two trace residues, wherein a correlation of greater than zero indicates that the trace residues have evolutionary ranks that are dependent on each other and a correlation of zero indicates that the trace residues have evolutionary ranks that are independent of each other;
aligning the traces from the protein sequence, wherein aligning is performed to maximize the evolutionary rank order correlation from each trace; and
determining a correlation between the two proteins with no sequence homology.

29. A method of determining a ligand binding pocket in a protein comprising the steps of:
determining global functional determinates of a family of proteins using quantitative evolutionary trace analysis, wherein determinates are residues that are involved in the global function of the protein;
obtaining protein sequences of a subfamily of proteins within the family having a common function;
aligning the protein sequences of the subfamily of proteins to generate a multiple sequence alignment;
producing an evolutionary trace, wherein the trace identifies residue that are trace residues; and
comparing the trace of the family to the trace of the subfamily, wherein a difference in the comparison yields the ligand binding pockets of the protein.

30. The method of claim 29, wherein the family of proteins is G-protein coupled receptors.

31. The method of claim 29, wherein the subfamily is Class A G-protein coupled receptors.

32. The method of claim 31, wherein the Class A G-protein coupled receptors are selected from the group consisting of opsins, adrenergic receptors, chemokine-related receptors and olfactory receptors.

33. The method of claim 29, wherein the subfamily is Class B G-protein coupled receptors.

34. The method of claim 33, wherein the Class B G-protein coupled receptors are secretin-related receptors.

35. A method of designing pharmaceuticals that target a protein comprising the steps of:
obtaining a protein sequence;
aligning the protein sequence to homologous protein sequences to generate a multiple sequence alignment;
predicting at least one residue in the protein sequence which is involved in the protein's function, wherein predicting the residues involves using quantitative evolutionary trace analysis; and
synthesizing the pharmaceutical to interact with the predicted residue in the protein.

36. The method of claim 35, wherein the pharmaceutical is a protein, a peptide or small molecule.

37. The method of claim 35 further comprising mutating at least one predicted residue prior to synthesizing the pharmaceutical.

38. The method of claim 37 wherein mutating produces an antagonist protein pharmaceutical.

39. The method of claim 37 wherein mutating produces an agonist protein pharmaceutical.

40. A method of designing pharmaceuticals that target a protein comprising the steps of:
obtaining a protein sequence;
aligning the protein sequence to homologous protein sequences to generate a multiple sequence alignment;
predicting at least one residue in the protein sequence which is involved in the protein's function, wherein predicting the residues involves using quantitative evolutionary trace analysis;
mutating at least one predicted residue in the protein sequence, wherein mutating modulates the protein's function; and
synthesizing the pharmaceutical to interact with the predicted residue in the protein.

41. The method of claim 40, wherein the pharmaceutical is a protein, a peptide or small molecule.

42. The method of claim 40, wherein modulates the protein's function is an enhancement of the binding of the protein to the target.

43. The method of claim 40, wherein modulates the protein's function is an interference with the binding of the protein to the target.

44. A method of determining the significance of a single nucleotide polymorphism in a protein, wherein the single nucleotide polymorphism occurs in a predicted trace residue comprising the steps of:
performing a quantitative evolutionary trace analysis on a protein;
performing a quantitative evolutionary trace analysis on a protein suspected of containing a single nucleotide polymorphism;
comparing the analysis on the protein to the analysis of the protein suspected of containing a single nucleotide polymorphism; and
assessing whether the single nucleotide polymorphism effects a residue that is predicted to be a functional site of the protein.

45. A method of screening compounds comprising the steps of:
obtaining a protein having predicted functional sites, wherein the functional sites are predicted using quantitative evolutionary trace analysis;
contacting the protein with a candidate substance;
determining whether the candidate substance interacts with the protein, wherein interaction with the protein indicates that the candidate substance is a ligand.

46. A method of determining a functional site in a nucleic acid sequence comprising the steps of:
obtaining a nucleic acid sequence;
aligning the sequence to homologous sequences to generate a multiple sequence alignment;
adding gap tolerance, wherein a gap in a sequence alignment is considered as an artificial nucleic acid;
producing an evolutionary trace, wherein the evolutionary trace identifies nucleic acids that are trace nucleic acids; and
determining cluster formation of the trace nucleic acids, wherein a cluster indicates the functional site of the nucleic acid sequence.

47. The method of claim 46, wherein determining comprises mapping the class-specific nucleic acid onto a representative structure and examining for cluster formation.

48. The method of claim 46, wherein obtaining comprises obtaining the nucleic acid sequence and homolog nucleic acid sequences.

49. The method of claim 39, wherein the nucleic acid sequence is DNA.

50. The method of claim 39, wherein the nucleic acid sequence is RNA.

51. The method of claim 46 further comprising determining clustering statistics.

52. The method of claim 51, wherein the clustering statistics comprises the overall number of clusters.

53. The method of claim 51, wherein the clustering statistics comprises the size of the largest cluster.

54. The method of claim 51, wherein the clustering statistics comprises the overall number of clusters and the size of the largest cluster.

55. A method of designing proteins with desired protein properties comprising the steps of:
obtaining a protein sequence;
aligning the protein sequence to homologous protein sequences to generate a multiple sequence alignment;
predicting at least one residue in the protein sequence which is involved in the protein's functions, wherein predicting the residues involves using quantitative evolutionary trace analysis;
synthesizing libraries of protein variants wherein residues at and/or around the predicted functional site are substituted with alternative amino acids; and
screening the resulting libraries for mutant proteins with the desired protein properties.

56. The method of claim 55, wherein the desired protein properties are selected from the group consisting of enhanced binding affinity, decreased immunogenicity, increased stability, increased solubility, decreased aggregation, and decreased crystallization.

57. The method of claim 55, wherein the desired protein properties are selected from the group consisting of decreased binding affinity, increased immunogenicity, decreased stability, decreased solubility, increased aggregation, and increased crystallization.

58. A designing a protein having altered protein properties comprising the steps of:
obtaining a protein sequence;
aligning the protein sequence to homologous protein sequences to generate a multiple sequence alignment;
predicting at least one residue in the protein sequence which is not related to the protein's function, wherein predicting the residues involves using quantitative evolutionary trace analysis;
synthesizing libraries of protein variants wherein at least one of the predicted residues are mutated to result in an altered protein property; and
screening the resulting libraries for mutant proteins with the desired altered protein properties.
